(19) United States
(12) Patent Application Publication
(10) Pub. No.: US 2026/0131140 A1
(43) Pub. Date: May 14, 2026

(54) MODIFYING EARLY-STAGE PARKINSON'S DISEASE PROGRESSION WITH DEEP BRAIN STIMULATION

(71) Applicant: VANDERBILT UNIVERSITY, Nashville, TN (US)

(72) Inventors: Philip David CHARLES, Nashville, TN (US); Mallory L. HACKER, Nashville, TN (US)

(21) Appl. No.: 19/118,598

(22) PCT Filed: Oct. 4, 2023
(86) PCT No.: PCT/US2023/075913

§ 371 Date: Apr. 4, 2025

Related U.S. Application Data
(60) Provisional application No. 63/413,760, filed on Oct. 6, 2022.

(51) Int. Cl. A61N 1/05; A61K 31/12; A61K 31/135; A61K 31/137; A61K 31/166; A61K 31/198; A61K 31/277; A61K 31/48; A61K 31/485; A61K 31/4985; A61N 1/02; A61N 1/36; A61P 25/16; G16H 20/30

(52) U.S. Cl. CPC A61N 1/0534; A61K 31/12; A61K 31/135; A61K 31/137; A61K 31/166; A61K 31/198; A61K 31/277; A61K 31/48; A61K 31/485; A61K 31/4985; A61N 1/025; A61N 1/36067; A61N 1/36135; A61P 25/16; G16H 20/30

(57) ABSTRACT

The present disclosure relates to the treatment of patients with early-stage Parkinson's Disease using subthalamic nucleus deep brain stimulation (STN-DBS) to a target a defined region of the brain. In particular, by positioning and/or programming the DBS electrode to stimulate cortical input fibers from the supplementary motor area (but not the pre-SMA), improved therapeutic benefits are obtained.

### PRIORITY CLAIM

This application claims benefit of priority to U.S. Provisional Application Ser. No. 63/413,760, filed Oct. 6, 2022, the entire contents of which are hereby incorporated by reference.

### BACKGROUND

### I. Field

The present disclosure relates to the fields of medicine, central nervous system disorders and neurobiology. More particularly, the disclosure relates to an improved method of performing subthalamic nucleus deep brain stimulation (STN-DBS) on a subject afflicted with early-stage Parkinson's Disease.

### II. Related Art

Parkinson's disease (PD) is a long-term degenerative disorder of the central nervous system that mainly affects the motor system. The symptoms generally come on slowly over time. Early in the disease, the most obvious are shaking, rigidity, slowness of movement, and difficulty with walking. Thinking and behavioral problems may also occur, and dementia becomes common in the advanced stages of the disease, as well as depression and anxiety (see in more than a third of PD patients). Other symptoms include sensory, sleep, and emotional problems. Thus, PD is a devastating disease with very limited treatment options; no cure is known.

Subthalamic nucleus deep brain stimulation (STN-DBS) is an established adjunctive therapy for mid- and advanced-stage Parkinson's disease (PD) that improves motor symptoms and quality of life as well as reduces medication burden and dyskinesia (Deuschl et al., 2006; Schuepbach et al., 2013). While many PD patients receive notable clinical benefit, individual motor response to DBS can be highly variable (ex: 3% to 63% improvement (Weaver et al., 2012)), and ˜25% of patients do not achieve a significant improvement in quality of life (Deuschl et al., 2006). Numerous groups have studied the source of this heterogenity, and patient factors such as younger age, shorter disease duration, and strong pre-operative response to levodopa predict good response to STN-DBS in advanced-stage PD (P. D. Charles et al., 2002; Welter et al., 2002).

Outside of patient characteristics, precise delivery of the intervention (i.e., electrode placement and subsequent programming) is also strongly associated with clinical outcome (Caire et al., 2013; Frizon et al., 2018; Horn, Li, et al., 2019; Neudorfer et al., 2022). Although there is not consensus on the optimal location or “sweet spot” (Blomstedt et al., 2018; Butson et al., 2006; Maks et al., 2008; Plaha et al., 2006), in recent years, the field seems to agree on optimal outcomes with active contacts within the dorsolateral (sensorimotor) STN (Akram et al., 2017; Bot et al., 2018; Caire et al., 2013; Horn, Li, et al., 2019) [for a review see (Horn, 2019)]. Furthermore, the STN receives input from numerous functional areas in the frontal cortex, and therefore, the site of stimulation will also determine the exact network modulated by DBS. While Parkinson's Disease has been considered a network disease since its discovery and neuromodulation has been targeting networks since the first days of electric brain stimulation (Hariz et al., 2010), the field is increasingly using advanced neuroimaging sequences to conceptualize DBS as a network treatment in individual patients, defining the networks as three-dimensional structures within stereotactic space (Akram et al., 2017; Horn, Reich, et al., 2017; Krauss et al., 2020; Lozano & Lipsman, 2013a; Sobesky et al., 2022). Moreover, from a bioelectrical perspective considering the tissue surrounding the electrode, effects arise from stimulating axons, not cell bodies, which may underline the increasing focus of the field on detemining white matter targets (Jakobs et al., 2019; Li et al., 2020). Associations between Parkinson's motor improvement and the hyperdirect, pallidofugal, and nigrofugal/striatofugal pathways suggest that precise localization stimulating specific fiber tracts (i.e., white matter tracts) connecting to relevant structures associated with motor control is extermely important for optimal symptomatic benefit of STN-DBS in PD (Akram et al., 2017; Avecillas-Chasin & Honey, 2020; Horn, Reich, et al., 2017). These recent DBS reports confirm early evidence from leisonal studies (Hassler et al., 1960) that cortical input from the supplementary motor cortex seems crucial, espeically when modulating hypokinetic symptoms (Akram et al., 2017; Horn, Li, et al., 2019; Horn, Reich, et al., 2017).

The robust improvements of STN-DBS in patients with mid- and advanced-stage PD motivate investigations into whether STN-DBS in very early-stage PD would extended or even enhance those benefits. Numerous preclinical studies suggest DBS intervention could be disease-modifying but only if applied early in the neurodegenerative process (Maesawa et al., 2004; Musacchio et al., 2017; A. L. Spieles-Engemann et al., 2010; Temel et al., 2006). With post-mortem evidence showing that 90% of dopaminergic innervation of the putamen is lost by 4 years after diagnosis (Kordower et al., 2013), there is growing acceptance that if DBS (or any potentially disease-modifying intervention) could slow PD progression, it would have to be applied at the very earliest stage of the disease (D. L. Fischer & Sortwell, 2019).

The first and, to date, only clinical trial to evaluate DBS at a stage with the potential to slow PD progression (early-stage PD: within four years of diagnosis, without history or evidence of dyskinesia or motor fluctuations) randomized 30 patients to bilateral STN-DBS plus optimal drug therapy (ODT) or ODT alone and followed them for two years (David Charles et al., 2014). In this trial, a 7-day washout of all PD medications and DBS stimulation, if applicable, was completed at baseline and every 6 months for 2 years. In lieu of a biomarker to track disease progression, the 7-day withdrawal of PD therapy allowed evaluation of underlying motor symptom progression without the overt influence of symptomatic therapies (i.e., STN-DBS and PD medications). All subjects who completed the 2-year trial enrolled in a 5-year follow-up visit which included ON therapy only evaluations at years 3, 4 and 5. A post-hoc analysis resulted in class II evidence that DBS in early-stage PD slows rest tremor progression (M. Hacker et al., 2018). Moreover, five-year outcomes provided class II evidence that DBS in early-stage PD decreases the risk of disese progression and polypharmacy (M. L. Hacker et al., 2020).

Thus, while the inventors have previously reported the ability to slow or stop tremor progression in some early-stage PD patients using STN-DBS, a better understanding of these results is needed in order to increase the number of patients that receive such benefits.

### SUMMARY

In accordance with the present disclosure, in one embodiment, there is provided a method of placing a deep brain stimulation (DBS) electrode into a patient having early-stage Parkinson's Disease (PD) comprising:
(a) mapping the patient's brain to determine a location for DBS electrode placement by identifying (i) fiber tracts from the supplementary motor area and/or primary motor area to the subthalamic nucleus (STN) of the patient; and (ii) fiber tracts from the pre-supplementary motor area to the STN of the patient; and
(b) implanting a DBS electode to target (a)(i) and avoid targeting (a)(ii).

Step (a) may comprise:
identifying the patient-specific location of the tracts defined in (a)(i) and (a)(ii) from a normative connectome by using inverse normalization to warp the tracts from the template space into the patient's brain space; or
identifying the patient-specific location of the tracts defined in (a)(i) and (a)(ii) from a normative connectome by normalizing the patient's brain to the template space which includes the tracts.

Step (a) may comprise utilizing patient-specific tractography data collected from diffusion-weighted brain imaging of the patient's brain to identify (a)(i) and (a)(ii) using the following regions of interest (ROI):
1) from the supplementary motor area projecting to the STN;
2) from the primary motor area projecting to the STN; and
3) from the pre-SMA projecting to the STN.
The method may further comprise performing a post-operative scan of the patient's brain.

In one embodiment, there is provided a method of placing and programming a deep brain stimulation (DBS) electrode into a patient having early-stage Parkinson's Disease (PD) comprising:
(a) implanting a DBS electode target the subthalamic nucleus (STN) of the patient;
(b) mapping the patient's brain to determine programming of said DBS electrode by identifying (i) fiber tracts from the supplementary motor area and/or primary motor area to the subthalamic nucleus (STN) of the patient; and (ii) fiber tracts from the pre-supplementary motor area to the STN of the patient, wherein the DBS electrode is programmed to stimulate (i) and avoid stimulating (ii).

Step (b) may comprise identifying the patient-specific location of the tracts defined in (b)(i) and (b)(ii) from a normative connectome by using inverse normalization to warp the tracts from the template space into the patient's brain space; or identifying the patient-specific location of the tracts defined in (b)(i) and (b)(ii) from a normative connectome by normalizing the patient's brain to the template space which includes the tracts.

Step (b) may comprise utilizing patient-specific tractography data collected from diffusion-weighted brain imaging of the patient's brain to identify (b)(i) and (b)(ii) using the following regions of interest (ROI):
1) from the supplementary motor area projecting to the STN;
2) from the primary motor area projecting to the STN; and
3) from the pre-SMA projecting to the STN.

The method may comprise determining whether said DBS electrode has achieved intended preoperative targeting of (b)(i) and non-targeting of (b)(ii).

The DBS electrode may comprise a plurality of contacts or segments and said method further comprises determining which contact(s) or segment(s) provide(s) the maximal stimulation of (b)(i) and avoids (b)(ii).

The DBS electrode may comprise a plurality of contacts or segments, and said method further comprises determining a field shape for said contact(s) or segment(s) that provide(s) maximal stimulation of (b)(i) and that avoids (b)(ii).

Any of the methods may further comprise treating said patient by delivering an electrical current through said DBS electrode, such as by continous delivery, patient modulated delivery, or by adaptive delivery based on patient parameters.

The patient may be a male human patient, a female human patient, or a non-human mammalian subject.

DBS may be performed more than once, such as, for example, on a chronic basis.

Any of the methods may further comprise treating said patient with a second PD therapy. The second PD therapy may be administered prior to, at the same time as, or after STN-DBS. The second PD therapy may be selected from levodopa, optionally in combination with a DOPA decarboxylase inhibitor (carbidopa, benserazide) or a COMT inhibitor (tolcapone, entacapone), a dopamine agonist (e.g., apomorphine, bromocriptine, pergolide, pramipexole, ropinirole, piribedil, cabergoline, apomorphine, lisuride), an MAO-B inhibitor (e.g., safinamide, selegiline, rasagiline), amantadine, an anticholinergics cholinesterase inhibitor, and lesional surgery, or combinations thereof.

The STN-DBS may result in one or more of slowing of motor symptom progression, stopping motor symptom progression, and/or reversing motor symptom progression. The STN-DBS may result in one or more of lower stimulation parameters, less need for post-operative dopaminergic medication, and/or less development of levodopa associated dyskinesia or other motor fluctuations.

In one embodiment, provided are Parkinson's Disease (PD) therapeutic agents for use in treating PD in a subject, wherein the subject separately, simultaneously or sequentially receives subthalamic nucleus (STN) deep brain stimulation (DBS) by a method as defined by any of the present embodiments.

The PD therapeutic agent may be administered prior to, at the same time as, or after STN-DBS.

The PD therapeutic agent may be levodopa, optionally in combination with a DOPA decarboxylase inhibitor (carbidopa, benserazide) or a COMT inhibitor (tolcapone, entacapone), a dopamine agonist (e.g., apomorphine, bromocriptine, pergolide, pramipexole, ropinirole, piribedil, cabergoline, apomorphine, lisuride), an MAO-B inhibitor (e.g., safinamide, selegiline, rasagiline), amantadine, and an anticholinergics cholinesterase inhibitor, or combinations thereof.

In one embodiment, provided herein are computer implemented methods for identifying deep brain stimulation (DBS) electrode placement locations for DBS treatment of a patient having early-stage Parkinson's Disease (PD) comprising the steps of:
(a) receiving brain image data for the patient;
(b) processing the brain image data to identify fiber tracts (i) from the supplementary motor area and/or primary motor area to the subthalamic nucleus (STN) of the patient, and (ii) from the pre-supplementary motor area to the STN of the patient; and
(c) generating an electrode placement map for the treatment of the patient using DBS, such that the implanted electrodes will target (a)(i) and avoid targeting (a)(ii).

In one embodiment, provided herein are computer implemented methods for identifying deep brain stimulation (DBS) electrode placement programming for DBS treatment of a patient having early-stage Parkinson's Disease (PD) comprising the steps of:
(a) receiving brain image data for the patient;
(b) processing the brain image data to identify fiber tracts (i) from the supplementary motor area and/or primary motor area to the subthalamic nucleus (STN) of the patient, and (ii) from the pre-supplementary motor area to the STN of the patient; and
(c) generating an electrode programming map for the treatment of the patient using DBS, such that the implanted electrodes will target (a)(i) and avoid targeting (a)(ii).

The computer implemented methods may further comprise identifying the patient-specific location of the tracts defined in (a)(i) and (a)(ii) from a normative connectome by using inverse normalization to warp the tracts from the template space into the patient's brain space. The computer implemented methods may further comprise utilizing patient-specific tractography data collected from diffusion-weighted brain imaging of the patient's brain to identify (a)(i) and (a)(ii) using the following regions of interest (ROI):
from the supplementary motor area projecting to the STN;
from the primary motor area projecting to the STN; and
from the pre-SMA projecting to the STN.
   The computer implemented methods may further comprise the steps of:
receiving postoperative brain image data for the patient; and
processing the postoperative brain image data to determine whether said DBS electrode has achieved the intended preoperative targeting of (a)(i) and non-targeting of (a)(ii).

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, for the method being employed to determine the value, or that exists among the study subjects. Such an inherent variation may be a variation of ±10% of the stated value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-C. Motor progression and therapeutic requirements of ‘top’ and ‘typical’ responding DBS+ODT subjects as compared to control subjects. A motor progression responder analysis using UPDRS-III 7-day OFF change from baseline to 24 months scores was used to split early DBS+ODT subjects into two groups: “top responders” (yellow, n=5; Δ≤0) and “typical responders” (blue, n=9; Δ>0). Early ODT control subjects are shown in grey (n=14). A subset of early DBS+ODT subjects (yellow lines) demonstrated slower motor progression (FIG. 1A) while requiring fewer PD medications (FIG. 1B) and lower stimulation amplitudes (FIG. 1C). Mean±SEM. Wilcoxon-rank sum P<0.05*top vs typical DBS+ODT responders, ^{#}top DBS+ODT responders vs ODT subjects.

FIGS. 2A-B. Anatomical distribution of DBS electrodes at the mesencephalic level. (FIG. 2A) Reconstructions of investigated leads in the early PD pilot trial cohort (n=14 subjects) are featured in the coronal plane. (FIG. 2B) 3D visualization of the STN features active electrode contacts mirrored onto the right hemisphere in the sagittal plane. Top responders, n=5 subjects (n=10 electrodes), yellow. Typical responders, n=9 subjects (n=18 electrodes), blue. STN: purple. The STN, derived from the DISTAL Minimal Atlas (Ewert et al. 2018), is superimposed on slices of a 100-μm, 7 T brain scan in MNI 152 space (Edlow et al. 2019).

FIGS. 3A-F. Sweet spot associated with slower motor progression in early-stage Parkinson's disease. (FIGS. 3A-C) Electric field vector magnitudes of all DBS subjects were rank-correlated with motor symptom progression scores in a voxel-wise manner. (FIG. 3A) Coronal, (FIG. 3B) axial, and (FIG. 3C) sagittal views are centered on the functional coordinates: 11.07±0.82 mm lateral, 1.83±0.61 mm posterior to, and 3.53±0.38 mm inferior to the midcommissural point (MNI peak coordinates: 11.4, −13.7, −7.6 mm). STN outlined in purple. Red nucleus outlined in red. Optimal located is noted by the white dashed line, the Bejjani line (Bejani et al., 2000). (FIGS. 3D-F) N-image of stimulation volumes showing broad coverage across the STN on a group level.

FIGS. 4A-E. White matter tracts associated with motor progression in early-stage Parkinson's disease. (FIG. 4A) The degrees of fibers modulated by E-fields were rank-correlated with slower motor progression scores across the entire DBS cohort (UPDRS-III 7-day OFF baseline to 24-month scores). Orange fibers (darker and more posterior) correlate positively with slower motor progression (R between 0.06 and 0.58), while cyan fibers correlate negatively (R between −0.53 and −0.01). Subthalamic nucleus (STN), purple. (FIG. 4B) Density maps of cortical fiber projections (positive/orange and negative/cyan) are overlayed onto an MNI space template [Johns Hopkins University (JHU) atlas parcellation: M1 (JHU: 25 & 26, precentral gyrus), SMA & pre-SMA (JHU: 1 & 2, superior frontal gyrus, posterior segment)] using Surf Ice software (world-wide-web at nitrc.org/projects/surfice). (FIG. 4C) Stimulation sites of top (green; #10) and poorly responding (red; #1) illustrative example subjects and their relationship to fibers associated with slowed motor progression (orange). (FIG. 4D) Top: Leave-one patient-out Cross-Validation of the fiber tract model shown in FIGS. 4A-C to estimate outcomes in unseen data. The analysis was iteratively repeated, each time leaving out one patient and estimating their outcomes by relating their stimulation site to the positively and negatively weighted fiber tracts. Repeating the same analysis in a 5-fold or 10-fold cross-validation also led to significant correlations (R=0.50, P=0.033 and R=0.53, P=0.027, respectively). Data from illustrative example subjects from FIG. 4C are featured. Bottom: distribution of motor progression scores by randomization group from the ‘DBS in early-stage PD’ pilot clinical trial. DBS, n=14. ODT, n=14. (FIG. 4E) The null-distribution of the leave-one-patient-out experiment from FIG. 4D (which was calculated by repeating the analysis 1,000 times after permuting motor progression values across subjects).

FIGS. 5A-F. Symptomatic Motor Improvement Sweet Spot and White Matter Tracts. (FIGS. 5A-C) Electric field magnitude values of all early-stage PD subjects were rank-correlated with percent symptomatic motor improvement (UPDRS-III MedON baseline to 24-month MedON/StimON scores), corresponding to the same analysis carried out with motor progression scores shown (FIGS. 3A-C). (FIG. 5A) Coronal and (FIG. 5B) axial views centered on the peak functional coordinates: 11.08±0.82 mm lateral, 1.93±0.60 mm posterior, and 3.48±0.38 mm inferior to the midcommissural point (MNI coordinates: 11.2, −13.7, −7.4 mm). STN outlined in purple. Red nucleus outlined in red. Bejjani line³²=white dashed line. (FIG. 5C) The degrees of fibers modulated by E-fields were rank-correlated with symptomatic motor improvement across the cohort (UPDRS-III ON baseline to 24-month scores), corresponding to the same analysis with motor progression scores shown in FIGS. 3A-C. Orange fibers are positively associated with symptomatic motor improvements (R between 0.00 and 0.59), cyan fibers show negative correlations (R between −0.53 and 0.00). (FIGS. 5D-F) Analysis of FIGS. 3A-C) repeated after regressing out symptomatic improvement (MedON/StimON) scores (as analyzed in FIGS. 5A-C) from motor progression scores.

FIGS. 6A-C. Spatial relationship between early-stage Parkinson's disease sweet spots and established landmarks in DBS targeting for Parkinson's disease. (FIG. 6A) Visualization of the optimal early PD locations (green spheres) associated with motor progression and symptomatic motor improvement and their relationship to mean coordinates derived from a meta-analysis of 342 standard of care PD electrodes (red sphere) (Caire et al. 2013). The mean Euclidean Distance between the metanalytic sweet spot and the sweet spots in the current study was 2.2±0.01 mm. Axial (FIG. 6B) and sagittal (FIG. 6C) sections featuring the peak coordinates in MNI space, with the Bejjani line drawn in red. STN functional regions: associative (blue), limbic (white), sensorimotor (orange). Red nucleus (red).

FIG. 7. Distinguishing Symptomatic versus Disease-Modifying Therapy. The red line represents relentless disease progression. The green line represents a treatment that completely stops disease progression but does not offer a cure. The gray line represents a therapy that is only symptomatic meaning that the symptom might be transiently improved for a short time but returns as soon as the therapy wears off (e.g., a dose of levodopa for PD bradykinesia). The dotted black line represents a therapy that is disease modifying—in this case slowing disease progression when applied in very early-stage Parkinson's disease. The therapy improves the symptoms when applied for a short time (symptomatic benefit) and also continues to provide benefit in slowing the progression of PD because the effect remains long after the therapy has been discontinued (i.e., disease modifying).

FIG. 8. Timing of DBS Intervention vs Nigrostriatal Degeneration (adapted from Fischer & Sortwell, 2019). Nigrostriatal degeneration [putaminal TH immunoreactivity (left Y-axis) and number of substantia nigra neurons (right Y-axis)] versus time since PD diagnosis (X-axis). Gray box indicates when other DBS trials were conducted (i.e., mid-stage PD, B; advanced-stage PD, C-E). The Vanderbilt DBS Trial (A) is the only trial conducted at a time when there are still nigrostriatal neurons and connections left to protect. It is therefore the only trial capable of modifying PD progression with DBS.

FIGS. 9A-C. Correlations Between Motor Progression and PD Therapies. (FIG. 9A) Slower motor progression correlates with lower stimulation amplitude (24-month voltage) for DBS subjects; R=−0.52, P=0.02. Amplitude shown as average of left and right leads. V=voltage. (FIG. 9B) Slower motor progression is also associated with greater reductions in LEDD after surgery for DBS subjects (black dots; R=−0.59, P=0.01). There is no correlation for ODT subjects (tan dots; R=0.16, P=0.54). (FIG. 9C) Stimulation sites of top (green) and poorly responding (red) illustrative example subjects. Stimulation and PD medication data from illustrative example DBS subjects from are featured in FIGS. 9A-B.

### DETAILED DESCRIPTION

As discussed above, subthalamic nucleus deep brain stimulation (STN-DBS) is an effective adjunctive therapy for mid- and advanced-stage Parkinson's disease (PD); however, individual motor improvement is variable, with electrode localization being a key determinant of efficacy. A DBS in early-stage PD pilot clinical trial randomized patients with early-stage PD 1:1 to optimal drug therapy (ODT) or bilateral STN DBS+ODT and included a 7-day therapeutic washout to evaluate progression of untreated motor symptoms of the 2-year trial. The objectives for the work described here were to: (1) investigate individual motor progression in the DBS in early PD trial and (2) explore the relationship between electrode localization and motor progression.

A post-hoc motor progression responder analysis was conducted using Unified Parkinson Disease Rating Scale Part III (UPDRS-III) 7-day OFF therapy scores (DBS+ODT n=14, ODT n=14). Voxel-wise probabilistic mapping and fiber tract connectivity (based on a normative connectome) were used to evaluate associations between electrode localization and clinical outcomes of motor progression (UPDRS-III 7-day OFF) and symptomatic motor improvement (UPDRS-III ON) in 14 subjects who received DBS in early-stage PD.

Untreated motor scores from baseline to 2 years worsened for all subjects randomized to ODT (14/14), while scores were unchanged (n=1) or improved (n=4) for one-third of subjects randomized to early DBS+ODT (5/14). Compared to the DBS+ODT group, the ODT group had over a three-fold increased chance of motor worsening. The location of stimulation most strongly associated with slowing motor progression was in the posteriorlateral STN (MNI coordinates: +11.25, −13.56, −7.44 mm). Fiber tracts projecting from supplementary motor area (SMA) and primary motor cortex (M1) to the STN positively correlated with slowed motor progression, whereas fiber tracts originating from pre-SMA and cerebellum were negatively associated with motor progression. An R-map model of stimulated fibers associated with change in motor progression was validated with leave-one-patient-out (R=0.56, P=0.02), 5-fold (R=0.50, P=0.03), and 10-fold (R=0.53, P=0.03) cross-validations. Sweet spot and fiber tract analyses that were repeated using symptomatic motor improvement scores showed strong similarities to motor progression location and connectivity.

These results suggest that stimulating the posteriorlateral STN in early-stage PD, specifically areas receiving input from M1 and SMA, can slow motor progression. A larger study will be conducted to further validate this finding, which study has already been approved by the FDA in a multicenter, phase 3 clinical trial evaluating DBS in early-stage PD.

These and other aspects of the disclosure are set forth below.

### I. PARKINSON'S DISEASE

Parkinson's disease (PD) is a long-term degenerative disorder of the central nervous system that mainly affects the motor system. The symptoms generally come on slowly over time. Early in the disease, the most obvious are shaking, rigidity, slowness of movement, and difficulty with walking. Thinking and behavioral problems may also occur. Dementia becomes common in the advanced stages of the disease. Depression and anxiety are also common occurring in more than a third of people with PD. Other symptoms include sensory, sleep, and emotional problems. The main motor symptoms are collectively called “parkinsonism”, or a “parkinsonian syndrome.”

The cause of Parkinson's disease is generally unknown but believed to involve both genetic and environmental factors. Those with a family member affected are more likely to get the disease themselves. There is also an increased risk in people exposed to certain pesticides and among those who have had prior head injuries while there is a reduced risk in tobacco smokers and those who drink coffee or tea. The motor symptoms of the disease result from the death of cells in the substantia nigra, a region of the midbrain. This results in not enough dopamine in these areas. The reason for this cell death is poorly understood but involves the build-up of proteins into Lewy bodies in the neurons. Diagnosis of typical cases is mainly based on symptoms, with tests such as neuroimaging being used to rule out other diseases.

There is currently no cure for Parkinson's disease. Initial treatment is typically with the anti-parkinsonian medication L-DOPA (levodopa), with dopamine agonists being used once levodopa becomes less effective. As the disease progresses and neurons continue to be lost, these medications become less effective while at the same time they produce a complication marked by involuntary writhing movements (i.e., dyskinesia). Diet and some forms of rehabilitation have shown some effectiveness at improving symptoms. Surgery to place microelectrodes for deep brain stimulation has been used to reduce motor symptoms in severe cases where drugs are ineffective. Evidence for treatments for the non-movement-related symptoms of PD, such as sleep disturbances and emotional problems, is less strong.

In 2013, PD was present in 53 million people and resulted in about 103,000 deaths globally. Parkinson's disease typically occurs in people over the age of 60, of which about one percent are affected. Males are more often affected than females. When it is seen in people before the age of 40 or 50, it is called young onset PD. The average life expectancy following diagnosis is between 7 and 14 years.

The term parkinsonism is used for a motor syndrome whose main symptoms are tremor at rest, stiffness, slowing of movement and postural instability. Parkinsonian syndromes can be divided into four subtypes, according to their origin (1) primary or idiopathic, (2) secondary or acquired, (3) hereditary parkinsonism, and (4) Parkinson plus syndromes or multiple system degeneration.

Parkinson's disease is the most common form of parkinsonism and is usually defined as “primary” parkinsonism, meaning parkinsonism with no external identifiable cause. In recent years several genes that are directly related to some cases of Parkinson's disease have been discovered. As much as this conflicts with the definition of Parkinson's disease as an idiopathic illness, genetic parkinsonism disorders with a similar clinical course to PD are generally included under the Parkinson's disease label. The terms “familial Parkinson's disease” and “sporadic Parkinson's disease” can be used to differentiate genetic from truly idiopathic forms of the disease.

Usually classified as a movement disorder, PD also gives rise to several non-motor types of symptoms such as sensory deficits, cognitive difficulties, and sleep problems. Parkinson plus diseases are primary parkinsonisms which present additional features. They include multiple system atrophy, progressive supranuclear palsy, corticobasal degeneration, and dementia with Lewy bodies.

In terms of pathophysiology, PD is considered a synucleinopathy due to an abnormal accumulation of alpha-synuclein protein in the brain in the form of Lewy bodies, as opposed to other diseases such as Alzheimer's disease where the brain accumulates tau protein in the form of neurofibrillary tangles. Nevertheless, there is clinical and pathological overlap between tauopathies and synucleinopathies. The most typical symptom of Alzheimer's disease, dementia, occurs in advanced stages of PD, while it is common to find neurofibrillary tangles in brains affected by PD. Dementia with Lewy bodies (DLB) is another synucleinopathy that has similarities with PD, and especially with the subset of PD cases with dementia. However, the relationship between PD and DLB is complex and still has to be clarified. They may represent parts of a continuum, or they may be separate diseases.

### A. Signs and Symptoms

Parkinson's disease affects movement, producing motor symptoms. Non-motor symptoms, which include autonomic dysfunction, neuropsychiatric problems (mood, cognition, behavior or thought alterations), and sensory and sleep difficulties, are also common. Some of these non-motor symptoms are often present at the time of diagnosis and can precede motor symptoms.

Four motor symptoms are considered cardinal in PD: tremor, rigidity, slowness of movement, and postural instability. Tremor is the most apparent and well-known symptom. It is the most common; though around 30% of individuals with PD do not have tremor at disease onset, most develop it as the disease progresses. It is usually a rest tremor—maximal when the limb is at rest and disappearing with voluntary movement and sleep. It affects to a greater extent the most distal part of the limb and at onset typically appears in only a single arm or leg, becoming bilateral later. Frequency of PD tremor is between 4 and 6 hertz (cycles per second). A feature of tremor is pill-rolling, the tendency of the index finger of the hand to get into contact with the thumb and perform together a circular movement. The term derives from the similarity between the movement of people with PD and the earlier pharmaceutical technique of manually making pills.

Bradykinesia is another characteristic feature of PD and is a slowness in the execution of movement. Performance of sequential and simultaneous movement is hindered. Initial manifestations are problems when performing daily tasks which require fine motor control such as writing, sewing or getting dressed. Clinical evaluation is based on similar tasks such as alternating movements between both hands or between both feet. Bradykinesia is not equal for all movements or times. It is modified by the activity or emotional state of the subject, to the point that some people are barely able to walk yet can still ride a bicycle. Generally, people with PD have less difficulty when some sort of external cue is provided.

Rigidity is stiffness and resistance to limb movement caused by increased muscle tone, an excessive and continuous contraction of muscles. In parkinsonism the rigidity can be uniform (lead-pipe rigidity) or ratchety (cogwheel rigidity). The combination of tremor and increased tone is considered to be at the origin of cogwheel rigidity. Rigidity may be associated with joint pain; such pain being a frequent initial manifestation of the disease. In early stages of Parkinson's disease, rigidity is often asymmetrical and it tends to affect the neck and shoulder muscles prior to the muscles of the face and extremities. With the progression of the disease, rigidity typically affects the whole body and reduces the ability to move.

Postural instability is typical in the late stages of the disease, leading to impaired balance and frequent falls, and secondarily to bone fractures. Instability is often absent in the initial stages, especially in younger people. Up to 40% may experience falls and around 10% may have falls weekly, with the number of falls being related to the severity of PD.

Other recognized motor signs and symptoms include gait and posture disturbances such as festination (rapid shuffling steps and a forward-flexed posture when walking), speech and swallowing disturbances including voice disorders, mask-like face expression or small handwriting, although the range of possible motor problems that can appear is large.

Parkinson's disease can cause neuropsychiatric disturbances, which can range from mild to severe. This includes disorders of speech, cognition, mood, behavior, and thought. Cognitive disturbances can occur in the early stages of the disease and sometimes prior to diagnosis and increase in prevalence with duration of the disease. The most common cognitive deficit in affected individuals is executive dysfunction, which can include problems with planning, cognitive flexibility, abstract thinking, rule acquisition, initiating appropriate actions and inhibiting inappropriate actions, working memory, and selecting relevant sensory information. Fluctuations in attention, impaired perception and estimation of time, slowed cognitive processing speed are among other cognitive difficulties. Memory is affected, specifically in recalling learned information. Nevertheless, improvement appears when recall is aided by cues. Visuospatial difficulties are also part of the disease, seen for example when the individual is asked to perform tests of facial recognition and perception of the orientation of drawn lines.

A person with PD has an increased risk of dementia compared to the general population. The prevalence of dementia increases with duration of the disease. Dementia is associated with a reduced quality of life in people with PD and their caregivers, increased mortality, and a higher probability of needing nursing home care.

Behavior and mood alterations are more common in PD without cognitive impairment than in the general population and are usually present in PD with dementia. The most frequent mood difficulties are depression, apathy and anxiety. Establishing the diagnosis of depression is complicated by symptoms that often occur in Parkinson's including dementia, decreased facial expression, decreased movement, a state of indifference, and quiet speech. Impulse control behaviors such as medication overuse and craving, binge eating, hypersexuality, or problem gambling can appear in PD and have been related to the medications used to manage the disease. Psychotic symptoms—hallucinations or delusions—occur in 4% of people with PD, and it is assumed that the main precipitant of psychotic phenomena in Parkinson's disease is dopaminergic excess secondary to treatment; it therefore becomes more common with increasing age and levodopa intake.

In addition to cognitive and motor symptoms, PD can impair other body functions. Sleep problems are a feature of the disease and can be worsened by medications. Symptoms can manifest as daytime drowsiness, disturbances in REM sleep, or insomnia. A systematic review shows that sleep attacks occur in 13.0% of patients with Parkinson's disease on dopaminergic medications.

Alterations in the autonomic nervous system can lead to orthostatic hypotension (low blood pressure upon standing), oily skin and excessive sweating, urinary incontinence and altered sexual function. Constipation and gastric dysmotility can be severe enough to cause discomfort and even endanger health. PD is related to several eye and vision abnormalities such as decreased blink rate, dry eyes, deficient ocular pursuit (eye tracking) and saccadic movements (fast automatic movements of both eyes in the same direction), difficulties in directing gaze upward, and blurred or double vision. Changes in perception may include an impaired sense of smell, sensation of pain and paresthesia (skin tingling and numbness). All of these symptoms can occur years before diagnosis of the disease.

### B. Causes

Parkinson's disease in most people is idiopathic (having no specific known cause). However, a small proportion of cases can be attributed to known genetic factors. Other factors have been associated with the risk of developing PD, but no causal relationships have been proven.

A number of environmental factors have been associated with an increased risk of Parkinson's, including pesticide exposure, head injuries, and living in the country or farming. Rural environments and the drinking of well water may be risks, as they are indirect measures of exposure to pesticides. Implicated agents include insecticides, primarily chlorpyrifos and organochlorines and pesticides, such as rotenone or paraquat, and herbicides, such as Agent Orange and ziram. Exposure to heavy metals has been proposed to be a risk factor, through possible accumulation in the substantia nigra, but studies on the issue have been inconclusive.

PD traditionally has been considered a non-genetic disorder; however, around 15% of individuals with PD have a first-degree relative who has the disease. At least 5% of people are now known to have forms of the disease that occur because of a mutation of one of several specific genes.

Mutations in specific genes have been conclusively shown to cause PD. These genes code for alpha-synuclein (SNCA), parkin (PRKN), leucine-rich repeat kinase 2 (LRRK2 or dardarin), PTEN-induced putative kinase 1 (PINK1), DJ-1 and ATP13A2. In most cases, people with these mutations will develop PD. With the exception of LRRK2, however, they account for only a small minority of cases of PD. The most extensively studied PD-related genes are SNCA and LRRK2. Mutations in genes including SNCA, LRRK2 and glucocerebrosidase (GBA) have been found to be risk factors for sporadic PD. Mutations in GBA are known to cause Gaucher's disease. Genome-wide association studies, which search for mutated alleles with low penetrance in sporadic cases, have now yielded many positive results.

The role of the SNCA gene is important in PD, because the alpha-synuclein protein is the main component of Lewy bodies. Missense mutations of the gene (in which a single nucleotide is changed), and duplications and triplications of the locus containing it have been found in different groups with familial PD. Missense mutations are rare. On the other hand, multiplications of the SNCA locus account for around 2% of familial cases. Multiplications have been found in asymptomatic carriers, which indicate that penetrance is incomplete or age dependent.

The LRRK2 gene (PARK8) encodes a protein called dardarin. The name dardarin was taken from a Basque word for tremor, because this gene was first identified in families from England and the north of Spain. Mutations in LRRK2 are the most commonly known cause of familial and sporadic PD, accounting for approximately 5% of individuals with a family history of the disease and 3% of sporadic cases. There are many mutations described in LRRK2, however unequivocal proof of causation only exists for a few.

Several Parkinson-related genes are involved in the function of lysosomes, organelles that digest cellular waste products. It has been suggested that some forms of Parkinson may be caused by lysosome dysfunctions that reduce the ability of cells to break down alpha-synuclein.

### C. Diagnosis

A physician will diagnose Parkinson's disease from the medical history and a neurological examination. There is no medical test that will clearly identify the disease, but brain scans are sometimes used to rule out disorders that could give rise to similar symptoms. People may be given levodopa and resulting relief of motor impairment tends to confirm the diagnosis. The finding of Lewy bodies in the midbrain on autopsy is usually considered proof that the person had Parkinson's disease. The progress of the illness over time may reveal it is not Parkinson's disease, and some authorities recommend that the diagnosis should be periodically reviewed.

Other causes that can secondarily produce a parkinsonian syndrome are Alzheimer's disease, multiple cerebral infarction and drug-induced parkinsonism. Parkinson-plus syndromes such as progressive supranuclear palsy and multiple system atrophy must be ruled out. Anti-Parkinson's medications are typically less effective at controlling symptoms in Parkinson-plus syndromes. Faster progression rates, early cognitive dysfunction or postural instability, minimal tremor or symmetry at onset may indicate a Parkinson-plus disease rather than PD itself. Genetic forms are usually classified as PD, although the terms “familial Parkinson's disease” and “familial parkinsonism” are used for disease entities with an autosomal dominant or recessive pattern of inheritance.

Medical organizations have created diagnostic criteria to ease and standardize the diagnostic process, especially in the early stages of the disease. The most widely known criteria come from the UK Parkinson's Disease Society Brain Bank and the U.S. National Institute of Neurological Disorders and Stroke. The PD Society Brain Bank criteria require slowness of movement (bradykinesia) plus either rigidity, resting tremor, or postural instability. Other possible causes of these symptoms need to be ruled out. Finally, three or more of the following features are required during onset or evolution: unilateral onset, tremor at rest, progression in time, asymmetry of motor symptoms, response to levodopa for at least five years, clinical course of at least ten years and appearance of dyskinesias induced by the intake of excessive levodopa. Accuracy of diagnostic criteria evaluated at autopsy is 75-90%, with specialists such as neurologists having the highest rates.

Computed tomography (CT) and conventional magnetic resonance imaging (MRI) brain scans of people with PD usually appear normal. These techniques are nevertheless useful to rule out other diseases that can be secondary causes of parkinsonism, such as basal ganglia tumors, vascular pathology and hydrocephalus. A specific technique of MRI, susceptibility weighted imaging has been found to differentiate between patients and subjects without the disease and another technique, diffusion MRI, has been reported to be useful at discriminating between typical and atypical parkinsonism, although its exact diagnostic value is still under investigation. Dopaminergic function in the basal ganglia can be measured with different PET and SPECT radioactive tracers. Examples are ioflupane (¹²³I) (trade name DaTSCAN) and iometopane (Dopascan) for SPECT or fluorodeoxyglucose (¹⁸F) and DTBZ for PET. A pattern of reduced dopaminergic activity in the basal ganglia can aid in diagnosing PD.

### D. Prevention, Management, Rehabilitation and Palliative Care

Exercise in middle age reduces the risk of Parkinson's disease later in life. Caffeine also appears protective with a greater decrease in risk occurring with a larger intake of caffeinated beverages such as coffee. Although tobacco smoke causes adverse health effects, decreases life expectancy and quality of life, it may reduce the risk of PD by a third when compared to non-smokers. The basis for this effect is not known, but possibilities include an effect of nicotine as a dopamine stimulant. Tobacco smoke contains compounds that act as MAO inhibitors that also might contribute to this effect.

Antioxidants, such as vitamins C and D, have been proposed to protect against the disease, but results of studies have been contradictory and no positive effect has been proven. The results regarding fat and fatty acids have been contradictory, with various studies reporting protective effects, risk-increasing effects or no effects. Also, there have been preliminary indications of a possible protective role of estrogens and anti-inflammatory drugs.

There is no cure for Parkinson's disease, but medications, surgery, and multidisciplinary management can provide relief from the symptoms. The main families of drugs useful for treating motor symptoms are levodopa (usually combined with a dopa decarboxylase inhibitor or COMT inhibitor that does not cross the blood-brain barrier), dopamine agonists and MAO-B inhibitors. The stage of the disease determines which group is most useful. Two stages are usually distinguished: an initial stage in which the individual with PD has already developed some disability for which he needs pharmacological treatment, then a second stage in which an individual develops motor complications related to levodopa usage. Treatment in the initial stage aims for an optimal tradeoff between good symptom control and side-effects resulting from improvement of dopaminergic function. The start of levodopa treatment may be delayed by using other medications such as MAO-B inhibitors and dopamine agonists, in the hope of delaying the onset of dyskinesias. In the second stage the aim is to reduce symptoms while controlling fluctuations of the response to medication. Sudden withdrawals from medication or overuse have to be managed. When medications are not enough to control symptoms, surgery and deep brain stimulation can be of use. In the final stages of the disease, palliative care is provided to improve quality of life.

Levodopa has been the most widely used treatment for over 30 years. L-DOPA is converted into dopamine in the dopaminergic neurons by dopa decarboxylase. Since motor symptoms are produced by a lack of dopamine in the substantia nigra, the administration of L-DOPA temporarily diminishes the motor symptoms.

Only 5-10% of L-DOPA crosses the blood-brain barrier. The remainder is often metabolized to dopamine elsewhere, causing a variety of side effects including nausea, dyskinesias and joint stiffness. Carbidopa and benserazide are peripheral dopa decarboxylase inhibitors, which help to prevent the metabolism of L-DOPA before it reaches the dopaminergic neurons, therefore reducing side effects and increasing bioavailability. They are generally given as combination preparations with levodopa. Existing preparations are carbidopa/levodopa (co-careldopa) and benserazide/levodopa (co-beneldopa). Levodopa has been related to dopamine dysregulation syndrome, which is a compulsive overuse of the medication, and punding. There are slow-release versions of levodopa in the form intravenous and intestinal infusions that spread out the effect of the medication. These slow-release levodopa preparations have not shown an increased control of motor symptoms or motor complications when compared to immediate release preparations.

Tolcapone inhibits the COMT enzyme, which degrades dopamine, thereby prolonging the effects of levodopa. It has been used to complement levodopa; however, its usefulness is limited by possible side effects such as liver damage. A similarly effective drug, entacapone, has not been shown to cause significant alterations of liver function. Licensed preparations of entacapone contain entacapone alone or in combination with carbidopa and levodopa.

Levodopa preparations lead in the long term to the development of motor complications characterized by involuntary movements called dyskinesias and fluctuations in the response to medication. When this occurs a person with PD can change from phases with good response to medication and few symptoms (“on” state), to phases with no response to medication and significant motor symptoms (“off” state). For this reason, levodopa doses are kept as low as possible while maintaining functionality. Delaying the initiation of therapy with levodopa by using alternatives (dopamine agonists and MAO-B inhibitors) is common practice. A former strategy to reduce motor complications was to withdraw L-DOPA medication for some time. This is discouraged now since it can bring dangerous side effects such as neuroleptic malignant syndrome. Most people with PD will eventually need levodopa and later develop motor side effects.

Several dopamine agonists that bind to dopaminergic post-synaptic receptors in the brain have similar effects to levodopa. These were initially used for individuals experiencing on-off fluctuations and dyskinesias as a complementary therapy to levodopa; they are now mainly used on their own as an initial therapy for motor symptoms with the aim of delaying motor complications. When used in late PD they are useful at reducing the off periods. Dopamine agonists include bromocriptine, pergolide, pramipexole, ropinirole, piribedil, cabergoline, apomorphine and lisuride.

Dopamine agonists produce significant, although usually mild, side effects including drowsiness, hallucinations, insomnia, nausea, and constipation. Sometimes side effects appear even at a minimal clinically effective dose, leading the physician to search for a different drug. Compared with levodopa, dopamine agonists may delay motor complications of medication use, but are less effective at controlling symptoms. Nevertheless, they are usually effective enough to manage symptoms in the initial years. They tend to be more expensive than levodopa. Dyskinesias due to dopamine agonists are rare in younger people who have PD, but along with other side effects, become more common with age at onset. Thus, dopamine agonists are the preferred initial treatment for earlier onset, as opposed to levodopa in later onset. Agonists have been related to impulse control disorders (such as compulsive sexual activity and eating, and pathological gambling and shopping) even more strongly than levodopa.

Apomorphine, a non-orally administered dopamine agonist, may be used to reduce off periods and dyskinesia in late PD. It is administered by intermittent injections or continuous subcutaneous infusions. Since secondary effects such as confusion and hallucinations are common, individuals receiving apomorphine treatment should be closely monitored. Two dopamine agonists that are administered through skin patches (lisuride and rotigotine) and are useful for people in the initial stages and possibly to control off states in those in the advanced state.

MAO-B inhibitors (safinamide, selegiline and rasagiline) increase the level of dopamine in the basal ganglia by blocking its metabolism. They inhibit monoamine oxidase B (MAO-B) which breaks down dopamine secreted by the dopaminergic neurons. The reduction in MAO-B activity results in increased L-DOPA in the striatum. Like dopamine agonists, MAO-B inhibitors used as monotherapy improve motor symptoms and delay the need for levodopa in early disease but produce more adverse effects and are less effective than levodopa. There are few studies of their effectiveness in the advanced stage, although results suggest that they are useful to reduce fluctuations between on and off periods. An initial study indicated that selegiline in combination with levodopa increased the risk of death, but this was later disproven.

Other drugs such as amantadine and anticholinergics may be useful as treatment of motor symptoms. However, the evidence supporting them lacks quality, so they are not first choice treatments. In addition to motor symptoms, PD is accompanied by a diverse range of symptoms. A number of drugs have been used to treat some of these problems. Examples are the use of quetiapine for psychosis, cholinesterase inhibitors for dementia, and modafinil for daytime sleepiness. A 2010 meta-analysis found that nonsteroidal anti-inflammatory drugs (apart from aspirin), have been associated with at least a 15 percent (higher in long-term and regular users) reduction of incidence of the development of Parkinson's disease.

Treating motor symptoms with surgery was once a common practice, but since the discovery of levodopa, the number of operations declined. Studies in the past few decades have led to great improvements in surgical techniques, so that surgery is again being used in people with advanced PD for whom drug therapy is no longer sufficient. Surgery for PD can be divided in two main groups: lesional and deep brain stimulation (DBS). Target areas for DBS or lesions include the thalamus, the globus pallidus or the subthalamic nucleus. Deep brain stimulation is the most commonly used surgical treatment, developed in the 1980s by Alim Louis Benabid and others. It involves the implantation of a medical device called a neurostimulator, which sends electrical impulses to specific parts of the brain. DBS is recommended for people who have PD with motor fluctuations and tremor inadequately controlled by medication, or to those who are intolerant to medication, as long as they do not have severe neuropsychiatric problems. Other, less common, surgical therapies involve intentional formation of lesions to suppress overactivity of specific subcortical areas. For example, pallidotomy involves surgical destruction of the globus pallidus to control dyskinesia.

Exercise programs are recommended in people with Parkinson's disease. There is some evidence that speech or mobility problems can improve with rehabilitation, although studies are scarce and of low quality. Regular physical exercise with or without physical therapy can be beneficial to maintain and improve mobility, flexibility, strength, gait speed, and quality of life. When an exercise program is performed under the supervision of a physiotherapist, there are more improvements in motor symptoms, mental and emotional functions, daily living activities, and quality of life compared to a self-supervised exercise program at home. In terms of improving flexibility and range of motion for people experiencing rigidity, generalized relaxation techniques such as gentle rocking have been found to decrease excessive muscle tension. Other effective techniques to promote relaxation include slow rotational movements of the extremities and trunk, rhythmic initiation, diaphragmatic breathing, and meditation techniques. As for gait and addressing the challenges associated with the disease such as hypokinesia (slowness of movement), shuffling and decreased arm swing; physiotherapists have a variety of strategies to improve functional mobility and safety. Areas of interest with respect to gait during rehabilitation programs focus on, but are not limited to improving gait speed, the base of support, stride length, trunk and arm swing movement. Strategies include utilizing assistive equipment (pole walking and treadmill walking), verbal cueing (manual, visual and auditory), exercises (marching and PNF patterns) and altering environments (surfaces, inputs, open vs. closed). Strengthening exercises have shown improvements in strength and motor function for people with primary muscular weakness and weakness related to inactivity with mild to moderate Parkinson's disease. However, reports show a significant interaction between strength and the time the medications was taken. Therefore, it is recommended that people with PD should perform exercises 45 minutes to one hour after medications when they are at their best. Also, due to the forward flexed posture, and respiratory dysfunctions in advanced Parkinson's disease, deep diaphragmatic breathing exercises are beneficial in improving chest wall mobility and vital capacity. Exercise may improve constipation.

One of the most widely practiced treatments for speech disorders associated with Parkinson's disease is the Lee Silverman voice treatment (LSVT). Speech therapy and specifically LSVT may improve speech. Occupational therapy (OT) aims to promote health and quality of life by helping people with the disease to participate in as many of their daily living activities as possible. There have been few studies on the effectiveness of OT and their quality is poor, although there is some indication that it may improve motor skills and quality of life for the duration of the therapy.

Palliative care is specialized medical care for people with serious illnesses, including Parkinson's. The goal is to improve quality of life for both the person suffering from Parkinson's and the family by providing relief from the symptoms, pain, and stress of illnesses. As Parkinson's is not a curable disease, all treatments are focused on slowing decline and improving quality of life and are therefore palliative in nature. Palliative care should be involved earlier, rather than later in the disease course. Palliative care specialists can help with physical symptoms, emotional factors such as loss of function and jobs, depression, fear, and existential concerns.

Along with offering emotional support to both the patient and family, palliative care serves an important role in addressing goals of care. People with Parkinson's may have many difficult decisions to make as the disease progresses such as wishes for feeding tube, non-invasive ventilator, and tracheostomy; wishes for or against cardiopulmonary resuscitation; and when to use hospice care. Palliative care team members can help answer questions and guide people with Parkinson's on these complex and emotional topics to help them make the best decision based on their own values.

Muscles and nerves that control the digestive process may be affected by PD, resulting in constipation and gastroparesis (food remaining in the stomach for a longer period than normal). A balanced diet, based on periodical nutritional assessments, is recommended and should be designed to avoid weight loss or gain and minimize consequences of gastrointestinal dysfunction. As the disease advances, swallowing difficulties (dysphagia) may appear. In such cases it may be helpful to use thickening agents for liquid intake and an upright posture when eating, both measures reducing the risk of choking. Gastrostomy to deliver food directly into the stomach is possible in severe cases.

Levodopa and proteins use the same transportation system in the intestine and the blood-brain barrier, thereby competing for access. When they are taken together, this results in a reduced effectiveness of the drug. Therefore, when levodopa is introduced, excessive protein consumption is discouraged and well-balanced Mediterranean diet is recommended. In advanced stages, additional intake of low-protein products such as bread or pasta is recommended for similar reasons. To minimize interaction with proteins, levodopa should be taken 30 minutes before meals. At the same time, regimens for PD restrict proteins during breakfast and lunch, allowing protein intake in the evening.

Repetitive transcranial magnetic stimulation temporarily improves levodopa-induced dyskinesias. Its usefulness in PD is an open research topic, although recent studies have shown no effect by rTMS. Several nutrients have been proposed as possible treatments; however, there is no evidence that vitamins or food additives improve symptoms. There is no evidence to substantiate that acupuncture and practice of Qigong, or Tai chi, have any effect on the course of the disease or symptoms. Further research on the viability of Tai chi for balance or motor skills are necessary. Fava beans and velvet beans are natural sources of levodopa and are eaten by many people with PD. While they have shown some effectiveness in clinical trials, their intake is not free of risks. Life-threatening adverse reactions have been described, such as the neuroleptic malignant syndrome.

PD invariably progresses with time. A severity rating method known as the Unified Parkinson's disease rating scale (UPDRS) is the most commonly used metric for clinical study. A modified version known as the MDS-UPDRS is also sometimes used. An older scaling method known as the Hoehn and Yahr scale (originally published in 1967), and a similar scale known as the Modified Hoehn and Yahr scale, have also been commonly used. The Hoehn and Yahr scale defines five basic stages of progression.

Motor symptoms, if not treated, advance aggressively in the early stages of the disease and more slowly later. Untreated, individuals are expected to lose independent ambulation after an average of eight years and be bedridden after ten years. However, it is uncommon to find untreated people nowadays. Medication has improved the prognosis of motor symptoms, while at the same time it is a new source of disability, because of the undesired effects of levodopa after years of use. In people taking levodopa, the progression time of symptoms to a stage of high dependency from caregivers may be over 15 years. However, it is hard to predict what course the disease will take for a given individual. Age is the best predictor of disease progression. The rate of motor decline is greater in those with less impairment at the time of diagnosis, while cognitive impairment is more frequent in those who are over 70 years of age at symptom onset.

Since current therapies improve motor symptoms, disability at present is mainly related to non-motor features of the disease. Nevertheless, the relationship between disease progression and disability is not linear. Disability is initially related to motor symptoms. As the disease advances, disability is more related to motor symptoms that do not respond adequately to medication, such as swallowing/speech difficulties, and gait/balance problems; and also to motor complications, which appear in up to 50% of individuals after 5 years of levodopa usage. Finally, after ten years most people with the disease have autonomic disturbances, sleep problems, mood alterations and cognitive decline. All of these symptoms, especially cognitive decline, greatly increase disability.

The life expectancy of people with PD is reduced. Mortality ratios are around twice those of unaffected people. Cognitive decline and dementia, old age at onset, a more advanced disease state and presence of swallowing problems are all mortality risk factors. On the other hand, a disease pattern mainly characterized by tremor as opposed to rigidity predicts an improved survival. Death from aspiration pneumonia is twice as common in individuals with PD as in the healthy population. In 2013 PD resulted in about 103,000 deaths globally, up from 44,000 deaths in 1990. The death rate increased from an average of 1.5 to 1.8 per 100,000 during that time.

### II. DISEASE MODIFYING THERAPY

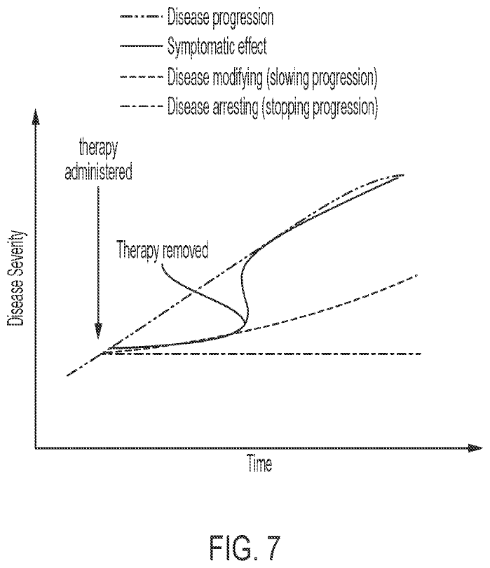

A significant advantage of the present disclosure is the provision of a disease modifying therapy (i.e., a therapy that can modify the progression of one or more features of the disease). This is distinct from symptomatic therapy that, while providing temporary relief from symptoms while the therapy is applied, does nothing to alter the course of the underlying disease itself. Put another way, in the case of a disease modifying therapy, the condition being studied does not worsen even after the therapy has been removed. As example of a symptomatic treatment with Parkinson's Disease tremor, when a patient takes a dose of levodopa the tremor is reduced for 2-3 hours and then returns (see FIG. 7 where therapy has been removed). As years go by, the degree of tremor continues to worsen and when a dose of medication has worn off, the patient suffers from ever worsening tremors. Specifically, in Parkinson's disease, patients receive less benefit from levodopa as the disease progresses and some patients have their tremor become completely resistant to levodopa and do not even receive the transient and temporary benefit seen shortly after taking a dose.

In the case of DBS therapy, this treatment, like levodopa, can reduce tremor symptomatically when the therapy is administered, i.e., the device is switched on. If the device is switched off after a brief time, however, the tremor returns to its underlying untreated severity (see Temperli et al., 2003). Here, the disclose method are (a) directed to people with only very early-stage Parkinson's disease, and (b) delivered such that specific nerve tracts are stimulated. This provides to early-stage PD patients a benefit on tremor progression (not just tremor symptoms) that remains present even after the DBS device is turned off—even turned off for a full week such that one is measuring the patient's tremor without any of the symptomatic (short term) benefit of DBS. These findings here permit a distinct application of DBS such that, in early-stage Parkinson's disease, the progression of the disease is slowed and, in some cases, can even be stopped.

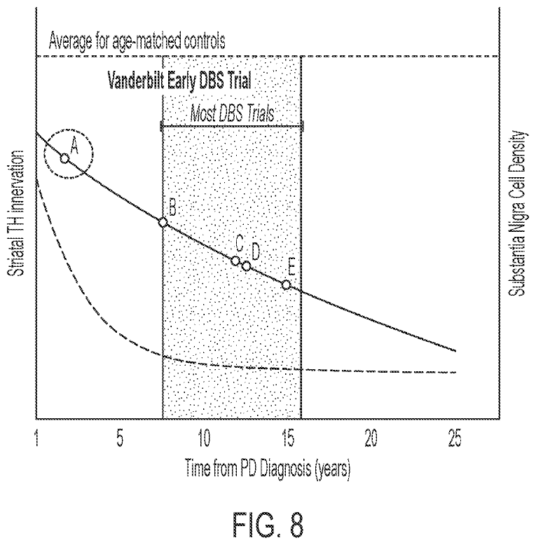

Preclinical studies demonstrate the importance of early DBS intervention (Maesawa et al., 2004; Musacchio et al., 2017; A. L. Spieles-Engemann et al., 2010; Temel et al., 2006), and the rationale to intervene early is further underscored by post-mortem data showing that 50% of putaminal denervation occurs by the time PD is diagnosed, rising sharply to 90% by 4 years disease duration (Kordower et al., 2013). Therefore, while numerous studies have evaluated the relationship between electrode placement and motor improvement in PD (Akram et al., 2017; Blomstedt et al., 2012; Bot et al., 2018; Butson et al., 2006; Caire et al., 2013; Horn, Li, et al., 2019; Maks et al., 2008; Plaha et al., 2006), this is the first study evaluating how stimulation location could affect disease progression. See FIG. 8.

Significantly, the overwhelming body of evidence in the literature confirms that tremor treated with VIM/DBS continues to relentlessly progress in the face of receiving such treatment (Shih et al., 2013; Favilla et al., 2012; Peters and Tisch, 2021; Fasano and Fasano, 2019; Paschen et al., 2019). Moreover, many patients with tremor even become resistant to VIM/DBS as their disease progresses (Favilla et al., 2012; Peters and Tisch, 2021; Fasano and Fasano, 2019). Thus, the disclosed methods are a substantial advance in the delivery of STN-DBS by providing a unique way of defining DBS lead placement and activation such long-term, disease modifying therapy can be reproducibly and accurately delivered.

### III. POSITIONING OF ELECTRODES IN SUBTHALAMIC NUCLEUS DEEP BRAIN STIMULATION (STN-DBS)

### A. Traditional Electrode Placement and Programming

Identification of the location for electrode placement is traditionally achieved as followed: 1) placement of bone fiducial markers, preoperative assessment to determine the patient's brain imaging, and preoperative target planning and trajectory assignment; and 2) post-operative testing of contacts and field shape for maximal effect.

The first procedure involving outpatient imaging and placement of bone fiducial markers, identifying operative targets, entry points, and landmarks is performed by a neurosurgeon.

During the second procedure by which the STN nucleus is mapped, a frame is affixed to the patient and tungsten microelectrodes (1 MΩ@1 kHz) are placed in guide tubes and advanced with electrode drives. Microelectrode recording (MER) is performed using a recording system. The microelectrodes are advanced toward the STN along the predefined trajectory. Recordings are made at regular intervals, beginning above the target and ending below the target or at the dorsal border of the substantia nigra pars reticularis (SNr). The recordings are interpreted based on accepted criteria by a neurophysiologist in the operating room and are used to define the borders of the STN and SNr. Determination of the optimal stimulation target was determined by consensus opinion of the neurosurgeon, neurologist, and neurophysiologist.

A general procedure for the identification of the STN nucleus is outlined in Starr (2002), which discloses “[t]he essential steps in DBS implantation are magnetic-resonance imaging (MRI)-guided stereotactic localization, confirmation of the motor territory of the target nucleus with microelectrode mapping, and intra-operative test stimulation to determine voltage thresholds for stimulation-induced adverse effects.” Details regarding methods for the identification of the STN nucleus as well as target selection is taught in Hutchinson et al. (1998), stating “[t]he STN can be identified by the presence of neurons with characteristic 25- to 45-Hz firing rates and irregular firing patterns, which may have movement- or tremor-related activity.”

Location selection is the topic of the review presented in Gross et al. (2006), as well as the rationale for employing physiological mapping in addition to standard imaging techniques in order to most accurately map the STN. The ideal locations for electrode placement are determined intraoperatively through microstimulation. Gross recites, “[m]icrostimulation at the site of site where tremor-related neurons were recorded can induce tremor arrest with a short latency . . . [and t]his effect is limited to specific body segments in accordance with the somatotopic arrangement. The use of a wider pulse duration (>0.5 ms) usually spreads the antitremor effect to other body regions after a longer delay (1-2 s).”

### B. Tractography-Based Electrode Placement and Programming

More recently, tractography-based surgical planning approaches are being used to leverage knowledge about associations between brain connectivity and clinical outcomes. Under this paradigm of “tractography-based” surgical planning; identification of the location for electrode placement is achieved as follows:
1) placement of bone fiducial markers, preoperative assessment to determine the patient's brain imaging, including incorporation of tractography data, and preoperative target planning and trajectory assignment; and
2) post-operative assessment of the patient's brain imaging to confirm lead placement and to identify contacts (conventional) or segments (directional) of the DBS electrode expected to provide good clinical outcome based on prior studies.
   The aforementioned Traditional Electrode Placement approach seeks to identify a location that provides symptomatic (i.e., transient, reversible in the absence of the therapy) benefit. The inventors introduce here an entirely novel tractography-based electrode placement and programming approach that aims to modify the progression of Parkinson's disease (i.e., slow, stop or reverse progression). Additional information needed for this novel approach includes:
1) placement of bone fiducial markers, preoperative assessment to determine the patient's brain imaging, including collecting tractography data, and preoperative target planning and trajectory assignment based on the tractography data, which includes identifying fiber tracts to (i) target (i.e., maximally stimulate) from the supplementary motor area (SMA) and/or primary motor area (M1) of the cortex to the STN and (ii) avoid from pre-SMA of the cortex to the STN; and
2) post-operative assessment of the patient's brain imaging to confirm lead placement and to identify contacts (traditional) or segments (directional) of the DBS electrode that (i) target (i.e., maximally stimulate) white matter fiber tracts from the supplementary motor area (SMA) and/or primary motor area (M1) of the cortex to the STN and (ii) avoid stimulating tracts from pre-SMA of the cortex to the STN.

Step 1, after bone fiducial markers are placed, preoperative assessment begins with performing a pre-operative MRI scan of the patient's brain. The next step—determining the location of lead placement to achieve the intended delivery and avoidance of delivery of electrical stimulation—can be performed using two different approaches: “patient-specific tractography” and “atlas-based tractography”. The first approach includes an additional pre-operative scan to collect a diffusion-weighted MRI of the patient's brain and using deterministic fiber tractography software to analyze the patient's brain scan to locate the relevant white matter fiber tracts. The second approach is to register (i.e., warp or normalize) the patient's pre-operative MRI brain scan with a brain “atlas” that has the fiber tracts previously identified (i.e., from a normative connectome in a prior study) visualized to predict the location of the relevant white matter fiber tracts. Once the target and avoidance white matter tracts are localized in a scan of the patient's brain, the “tractography-based surgical planning” is completed by providing the neurosurgeon with the tractography output (i.e., imaging file) that is incorporated into standard target planning software such as Brainlab Elements (Brainlab AG, Munich, Germany) or StealthStation FrameLink (Medtronic, USA) to place the electrode in a position that will optimally stimulate positive fiber tracts and avoid negative fiber tracts.

The first embodiment may be referred to as “patient-specific tractography” where pre-operative patient scans are analyzed by software to map the relevant white matter fiber tracts. Deterministic tractography (“fiber tracking”) is performed based on the diffusion-weighted (DWI) scans collected preoperatively. The patient's DWI brain scans are co-registered to the patient's structural (i.e., T1, T2) brain scans. Regions of interest (ROIs) are identified on the structural brain scans and to establish the start and finish of the desired white matter tract. Numerous publications describe this established methodology, including Graat et al., 2022, Riva-Posse et al., 2017, and Noecker et al., 2018. For the methods specified in this application, white matter tracts from the following ROI pairs are needed for surgical planning: tracts to target (M1 to STN, SMA to STN), tracts to avoid (pre-SMA to STN).

The second embodiment may be referred to as “atlas-based tractography.” A variety of software packages allow the user to visualize the reference “atlas” brain and then perform the “warping” or normalization into a patient's brain scan (and vice versa, known as an “inverse transformation” or “reverse normalization”), including ANTs Rigid/Affine (Ashburner, 2007), BRAINSFIT (Johnson et al., 2007), SPM Co-register (Friston et al., 2004), FSL FLIRT (Jenkinson et al., 2002), Hybrid SPM/ANTs, Hybrid SPN/FSL and Hybrid SPM/BRAINSFIT. Another example is the Lead-DBS toolbox reported by Ewert et al. (2019) which uses an “effective low variance+subcortical refinement” preset of the ANTS SyN algorithm that are highly optimized for nonlinearly registering subcortical elements with submillimeter precision. The Lead-DBS toolbox was originally developed at Charite—University of Medicine (CCM), Berlin, Germany (Horn & Kuhn, NeuroImage, 107:127-135, 2015). This “atlas-based tractography” methodology is described by Oxenford et al., 2022 using the Lead-DBS, Lead-Group, Lead-OR software framework which supports integration with planning software (Brainlab Elements, Brainlab AG, Munich, Germany) and the NeuroOmega system (Alpha Omega Engineering). For the invention specified in this application, specific white matter fiber tracts identified from a normative connectome (described below, Example 1), which are in ICBM 2009b NLIN asymmetric (“MNI”) space (Fonov et al., 2011) template space, are warped into the patient's pre-operative MRI scan to allow for the visualization of these fiber tracts in “patient space” (i.e., native space) needed for surgical planning.

Postoperative assessment includes collecting an additional scan of the patient's brain (e.g., CT or MRI) that permits visualization of the implanted DBS electrodes. To facilitate DBS programming based on the target and avoidance white matter fiber tracts (e.g., “tractography-based DBS programming”), the patient's post-operative brain scans are co-registered to the patient's pre-operative structural scans which contain the target and avoidance tracts, which can be identified through either method described above). Probabilistic software, such as the Lead-DBS or CranialVault/CranialCloud™ suites, is used to reconstruct the DBS electrode and subsequently visualize the white matter tract activation based on the contact or segments of the electrode that are active.

### C. Stimulation Paradigms

Once the electrode placement and contact selection is complete, as described above, the device is then activated so that stimulation therapy is given to the patient. Conventional deep brain stimulation (cDBS) systems are “open-loop” such that high-frequency stimulation settings are typically unchanged between clinic visits with the programming physician. Recently, a new type of DBS stimulation has been introduced which adapts to signals received from the patient. This type of stimulation is currently investigational and also known as “closed-loop” or adaptive DBS (aDBS). Adaptive DBS turns stimulation on or off by responding to patient data, which can include physiological signals (i.e., beta band signals detected from “sensing” DBS systems, such as Medtronic Percept) or movement-based signals (i.e., patient motion detected from a wearable device). Efficacy for conventional DBS is well-established (Deuschl et al., 2006; Schuepbach et al., 2013), while clinical trials evaluating efficacy for closed-loop/adaptive DBS are ongoing (NCT04547712).

### IV. COMBINATION TREATMENTS

It also may prove advantageous to use combination therapies in the treatment of PD, where a therapy is added to the STN-DBS therapy. Such therapies may, in combination, provide better results that the individual therapies, and in some cases, may provide more than additive effects. In other cases, they may reduce the amount of one or the other therapies required to achieve clinical benefit.

This process may involve administering both therapies at the same time. Alternatively, the STN-DBS therapy may precede or follow the other treatment by intervals ranging from minutes to weeks. In embodiments where the other therapy and STN-DBS are applied separately to the subject, one would generally ensure that a significant period of time did not expire between each delivery, such that the other therapy and STN-DBS would still be able to exert an advantageously combined effect on the subject. In such instances, it is contemplated that one may administer both modalities within about 12-24 h of each other and, more preferably, within about 6-12 h of each other. In some situations, it may be desirable to extend the time period for treatment significantly, however, where one or several days (2, 3, 4, 5, 6 or 7) to several weeks (1, 2, 3, 4, 5, 6, 7 or 8) lapse between the respective administrations.

Various combinations may be employed; for example, the STN-DBS therapy is “A” and the second PD therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of the therapies to a patient will follow general protocols for the administration of that particular secondary therapy, taking into account the toxicity/side effects, if any, of the treatments. It is expected that the treatment cycles would be repeated as necessary.

As discussed above, while there is no cure for Parkinson's disease, medications, surgery, and multidisciplinary management can provide relief from the symptoms. These therapies include levodopa (usually combined with a DOPA decarboxylase inhibitor like carbidopa and benserazide or a COMT inhibitor, such as tolcapone or entacapone, that does not cross the blood-brain barrier), dopamine agonists (e.g., apomorphine, bromocriptine, pergolide, pramipexole, ropinirole, piribedil, cabergoline, apomorphine and lisuride), MAO-B inhibitors (e.g., safinamide, selegiline and rasagiline), amantadine, anticholinergics cholinesterase inhibitors, and lesional surgery.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Methods

Parkinson's Disease Cohort. This retrospective study evaluated subjects from the DBS in the early PD pilot clinical trial (NCT00282152; IDEG050016; Vanderbilt IRB #040797). Trial design (D Charles et al., 2012), operative and surgical targeting experiences (Camalier et al., 2014; E Kahn et al., 2011), 2-year (David Charles et al., 2014) and 5-year (M. L. Hacker et al., 2020) results, and post hoc analyses (M. Hacker et al., 2018; M. L. Hacker et al., 2015) were previously reported. Briefly, 30 early-stage PD patients were randomized 1:1 to receive bilateral STN-DBS plus ODT or ODT alone for 2 years. Key enrollment criteria included PD medication 6 months to 4 years, Modified Hoehn & Yahr Stage II off medication, and no history or evidence of dyskinesia or motor fluctuations. Fifteen subjects randomized to early STN-DBS plus ODT were implanted bilaterally with quadripolar DBS electrodes (model 3389, Medtronic, Minneapolis, MN). One early DBS+ODT subject was excluded from this analysis because of missing data (baseline Unified Parkinson's Disease Rating Scale Part II (UPDRS-III) 7-day off score and 24-month stimulation parameters for the right electrode which was explanted prior to 24-month assessment).

Therapeutic Washout and Clinical Assessments. During the 2-year trial, subjects were admitted to the Vanderbilt Clinical Research Center for a 7-day washout of all PD therapies at baseline, 6-, 12-, 18- and 24-months. At baseline, the UPDRS-II motor examination was videotaped in the ON and OFF therapy states: ON therapy (day 1; ON medication) and 7 days OFF therapy (day 8; OFF medication). At subsequent study visits, the UPDRS-III motor examination was again videotaped in the ON and OFF therapy states: ON therapy (day 1; ON medication and ON stimulation, if applicable) and 7 days OFF therapy (day 8; OFF medication and OFF stimulation, if applicable). After the trial completed, videotapes were scored by an independent rater blinded to treatment assignment, ON versus OFF therapy status, and study visit sequence. Baseline UPDRS 7-day OFF scores were used to calculate PD phenotype (i.e., tremor-dominant (TD), postural instability/gait difficulty (PIGD)), following previously reported methods (M. Hacker et al., 2018; Stebbins et al., 2013).

Therapeutic Management. Subjects' treating neurologists managed medication and stimulation parameters. All DBS+ODT subjects were treated using monopolar stimulation with case positive and a single optimal contact negative (David Charles et al., 2014). Beginning four weeks postoperatively, the optimal contact was programmed at 130 Hertz (Hz) and 60 psec pulse width. Levodopa equivalent daily doses (LEDD) were calculated as previously described (Tomlinson et al., 2010).

DBS Electrode Localizations. Preoperative T1 and T2 MRI scans and postoperative CT scans were acquired (Camalier et al., 2014). The advanced processing pipeline in Lead-DBS was used to localize electrodes (lead-dbs.org; (Horn & Kuhn, 2015)). Postoperative CTs were linearly coregistered to preoperative MRI using advanced normalization tools (ANTs; stnava.github.io/ANTs/; (Brian B. Avants et al., 2011)), and subsequent coregistrations were inspected and refined if needed. The brain shift correction step from Lead-DBS was applied. All preoperative volumes were normalized to ICBM 2009b NLIN asymmetric (“MNI”) space (Fonov et al., 2011) applying the ANTs SyN Diffeomorphic Mapping (Avants et al., 2008) using the preset “effective: low variance default+subcortical refinement”. This method was top-performer to segment the STN with precision comparable to manual expert segmentations in a recent comparative study (Ewert et al., 2018). DBS electrodes were automatically pre-reconstructed using the phantom-validated and fully-automated PaCER method (Husch et al., 2018) and manually refined if needed. Atlas segmentations in this study were defined by the DISTAL atlas (Ewert et al., 2018). Group visualizations were performed using the Lead Group toolbox (Treu et al., 2020).

E-field Modeling. Electric field vector magnitudes (the term E-fields will be used as shorthand for the purpose of this manuscript) were used to estimate the volume of tissue modulated around the electrodes. E-fields were calculated based on the 24-month DBS programming settings applied using an adaptation of the SimBio/FieldTrip pipeline (Vorwerk et al., 2013) as implemented in Lead-DBS (Horn, Li, et al., 2019). E-fields were nonlinearly flipped to the contralateral side since no asymmetric effects were assumed, resulting in 2×14=28 E-fields across the cohort.

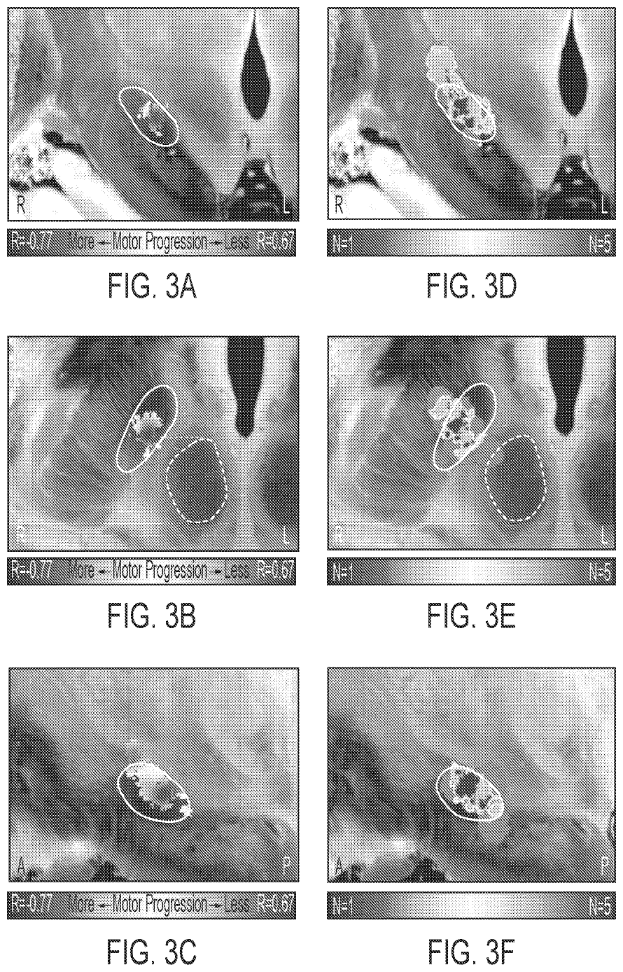

DBS Sweet Spot Mapping. Sweet spots associated with clinical outcomes (motor symptom progression (UPDRS-III 7-day OFF change from baseline to 24 months); symptomatic motor improvement (UPDRS-III ON percent change from baseline to 24 months)) were assessed using Lead-Group (Treu et al., 2020). For each voxel covered by the group of E-fields across the cohort in MNI space, E-field vector magnitudes across subjects were Spearman rank-correlated with the two clinical outcome variables (motor progression and motor improvement). The area of interest was conservatively restricted to voxels that were covered by at least 20% of E-fields with a vector magnitude above 0.2 V/m (a typical value assumed for DBS to activate axons (═ström et al., 2015)). For visualization, sweet spots were smoothed using a full-width-half-maximum kernel of 2 mm, while rank-correlation coefficients in color bars of FIGS. 3A-F and 5A-F were derived from unsmoothed files.

DBS Fiber Filtering. Fiber tract connectivity was assessed using a connectome modified from the DBS Tractography Atlas (Middlebrooks et al., 2020) to include additional connections from cortex to STN and from STN to substantia nigra pars compacta and pars reticulata (Supplemental Methods). For the finite set of 6,525,876 fiber tracts represented in the resulting Netstim Tractography Atlas and each subject's E-field, a value of probabilistic impact on the tract was calculated as previously described (Horn et al., 2022). Tracts were considered connected if the mean E-field magnitude they traverse was >1000 V/m and if they were connected to >5% of E-fields.

Statistical Analysis. A motor progression responder analysis was conducted whereby each subject was categorized based on their post-washout change (A) in UPDRS-III 7-day OFF motor score from baseline to two years as improved (Δ<0), no change (Δ=0), or worsened (Δ>0). Fisher's exact test was used to assess the difference between the ODT and DBS+ODT groups in the risk of motor score worsening (worsened vs. improved or no change). The difference between the two groups in the trend toward worsening was assessed using exact logistic regression of the ordered outcome scores (improved, no change, worsened); the logistic model included the treatment group as the outcome (1=ODT, 0=DBS+ODT) and the change in motor score from baseline to two years as an ordered score (improved=0, no change=1, worsened=2) as the only explanatory variable. The difference in trend towards worsening was assessed using the estimated odds ratio for the ordered score. Wilcoxon rank-sum tests were used to compare mean stimulation amplitude between top and typical DBS+ODT responders at each follow-up visit and LEDD change from baseline between top and typical DBS+ODT responders and between top DBS+ODT responders and ODT subjects. Analyses of clinical data were conducted in SAS 9.3 (SAS Institute Inc, Cary, NC) and STATA 17.0 (StataCorp LP, College Statio, TX).

Strength of structural connectivity was Spearman rank-correlated with change in motor progression (baseline to 24 months) which yielded a connectivity map showing positive and negative tract associations with motor progression or with motor improvement (i.e., R-maps). In other words, Spearman's rank correlation coefficients showing positive values for tract populations maximally associated with electrodes in top responding subjects and negative values for the ones modulated in poor responding subjects. Significance (at a P=0.05 level) was tested using out-of-sample data (leave-one-patient out, 5-fold and 10-fold cross-validations).

### Example 2—Results

Demographics and baseline characteristics of subjects randomized in the DBS in early-stage PD pilot clinical trial are presented in Table 1. Clinical results are described in detail elsewhere, (David Charles et al., 2014; M. L. Hacker et al., 2020). Briefly, the DBS+ODT cohort comprised 14 patients (13 male, mean baseline age 60.9±6.9 years) who were operated on in early-stage PD (mean baseline disease duration 2.6±1.9 years).

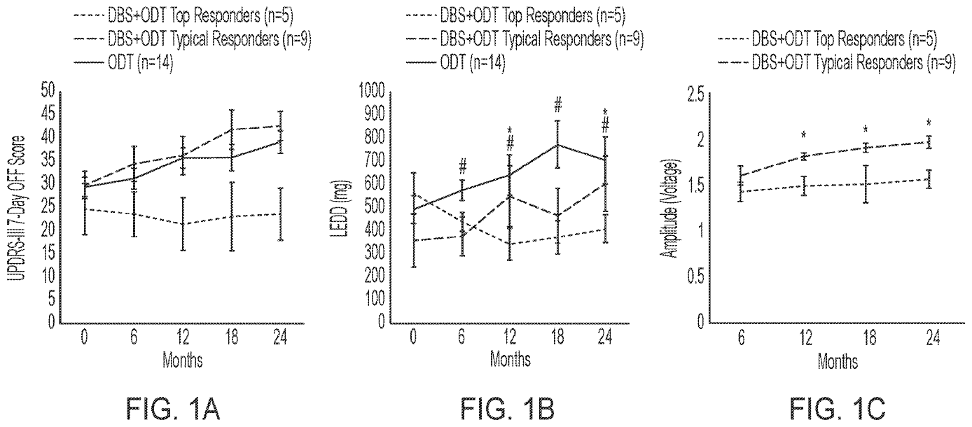

Motor Progression Responder Analysis. A responder analysis was conducted to evaluate motor symptom progression from baseline to 24 months. As expected, UPDRS-III 7-day OFF medication motor scores worsened over 2 years for all subjects randomized to ODT (14/14). In contrast, UPDRS-III 7-day OFF medication/OFF stimulation motor scores remained stable or improved in five subjects randomized to early DBS+ODT (5/14, FIG. 1A). Compared to the DBS+ODT group, the ODT group had over a three-fold increased chance of motor worsening (exact logistic regression median unbiased estimated odds ratio, ODT vs. ODT+DBS=3.68, P=0.04). To investigate DBS-related differences in the context of electrical stimulation, the DBS+ODT cohort was split into two groups based on UPDRS-III OFF change scores: “top responders” (n=5; UPDRS-III OFF Δ<0) and “typical responders” (n=9; UPDRS-III OFF Δ>0). Among top responders, UPDRS-III 7-day OFF scores at 2 years were improved from baseline for 4 out of the 5 subjects and remained unchanged for the fifth subject. Demographics and baseline characteristics for the top and typical DBS+ODT responders are featured in Table 1.

Reduced Medications and Stimulation. In the DBS+ODT cohort, top responders (n=5; UPDRS-III OFF Δ≤0) required less medication on average at each follow-up visit as compared to baseline (FIG. 1B). Mean LEDD change from baseline to 24 months for the DBS+ODT top responders (−148±227 mg) was significantly lower as compared to the DBS+ODT typical responders (245±357 mg) and the ODT subjects (215±360 mg; P=0.04, P=0.03, respectively). Mean stimulation voltage was also lower for the early DBS+ODT top responders than the typical responders, and this difference was significant at 12-, 18-, and 24-months (P<0.01, P=0.04, P=0.03), respectively (FIG. 1C). At 24 months, top DBS+ODT responders had mean stimulation voltage of 1.6±0.3 V, whereas typical DBS+ODT responders had mean stimulation voltage of 2.0±0.2 V. In other words, this subgroup of early DBS+ODT subjects required fewer medications and lower stimulation amplitudes while demonstrating slower motor progression. To determine whether differences in stimulation site across the DBS+ODT cohort was able to explain the observed variance in clinical outcome and therapeutic requirements, a voxel-wise probabilistic stimulation mapping and structural connectivity analysis was performed.

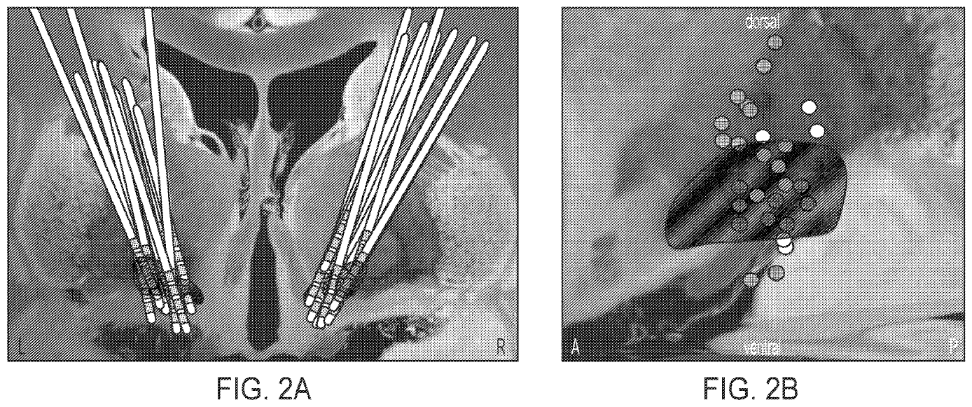

Relationship Between Motor Progression and Stimulation Site. DBS Sweet Spot Analysis. Electrode localization revealed placement of active contacts within the STN and surrounding eloquent areas in all subjects (FIGS. 2A-B), as previously reported (Elyne Kahn et al., 2012). E-fields for all early DBS+ODT subjects were used to identify the regions associated with change in motor symptom progression (FIGS. 3A-C). The aggregate volume derived from voxel-wise probabilistic mapping revealed the strongest slowing of motor progression in the posterolateral aspect of the motor portion of STN (FIGS. 3A-C). The peak location (i.e., the location most strongly associated with slowing of motor progression) was located at the following MNI coordinates: +11.25, −13.56, −7.44 mm (Spearman's rank correlation coefficient at peak: R=0.67). Expressed in functional (AC/PC) coordinates (Horn, Kuhn, et al., 2017), this site maps to 11.07±0.82 mm lateral, 1.83±0.61 mm posterior, and 3.53±0.38 mm inferior to the midcommissural point. In contrast, impingement of more anterior and dorsal regions that primarily encompassed zona incerta, was associated with a greater degree of motor progression.

The peak location (i.e., the location most strongly associated with the least amount of motor progression) was located at MNI coordinates: +11.25, −13.56, −7.44 mm (Spearman's rank correlation coefficient at peak: R=0.67). Expressed in functional (AC/PC) coordinates (Horn, Kühn et al., 2017), this maps to 11.07±0.82 mm lateral, 1.83±0.61 mm posterior, and 3.53±0.38 mm inferior to the midcommissural point. In contrast, involvement of more anterior and dorsal regions that primarily encompassed zona incerta, was associated with a greater degree of motor progression. The N-map of stimulation volumes covered a larger area encompassing the entire motor STN (FIGS. 3D-F).

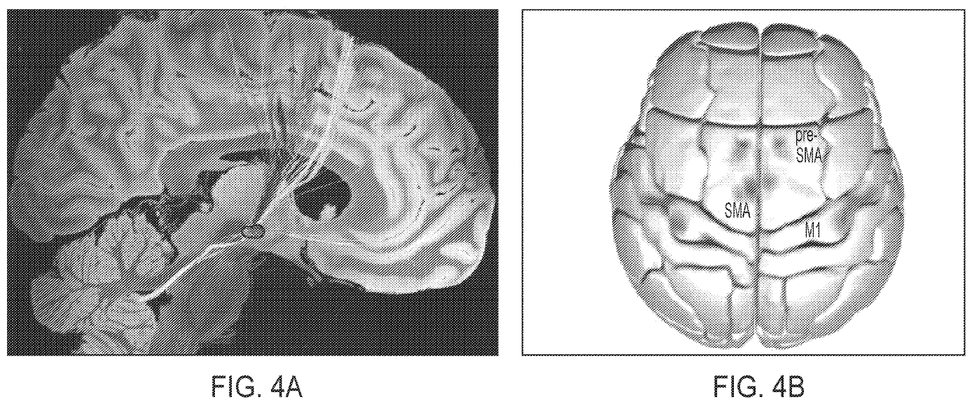

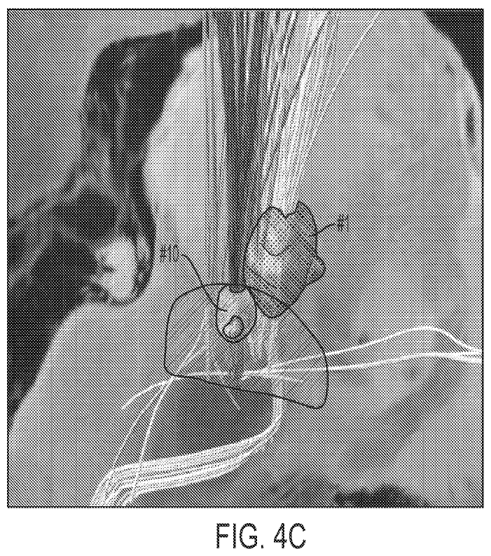

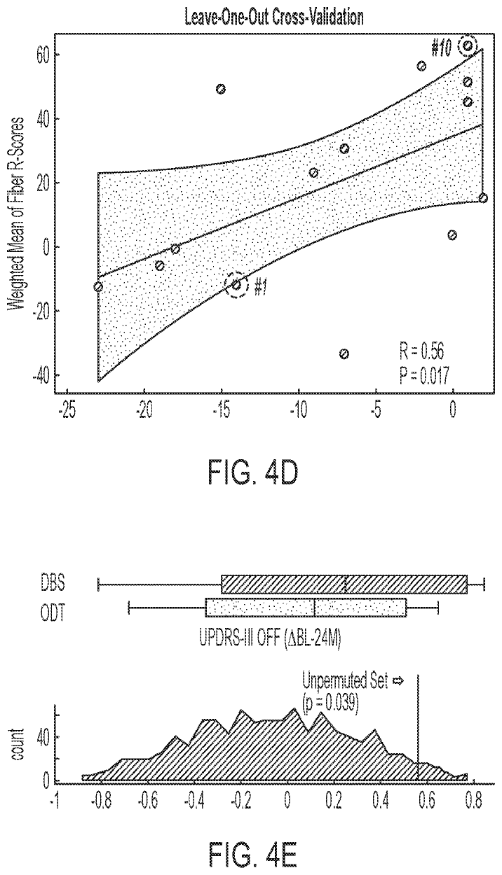

DBS Fiber Filtering Analysis. To interrogate the structural networks implicated in motor progression, E-fields derived from the DBS+ODT cohort were used to seed from a structural connectome (Netstim Tractography Atlas). Rank-correlation of E-field magnitudes with motor progression scores revealed distinct fiber tracts associated with contrasting clinical outcome (FIG. 4A). Specifically, positively correlated fibers projected from supplementary motor area (SMA) and primary motor cortex (M1) to the posterior aspect of STN (FIGS. 4A-B), In contrast, negatively correlated fibers originated from pre-SMA and cerebellum, reaching more anterior aspects of STN with the sensorimotor/associative transition zone and posterior subthalamic area (PSA), respectively (FIG. 4A).

Tracts were weighted by the degree of how much their modulation correlated with motor progression across the DBS cohort. This model was validated using leave-one-patient-out (FIG. 4D, R=0.56, P=0.02), 5-fold (R=0.50, P=0.03), and 10-fold (R=0.53, P=0.03) cross-validations. To investigate the relative contribution of positive and negative tracts, analyses were repeated using only positive tracts (leave-one-patient-out CV: R=0.48, P=0.05), 5-fold CV: (R=0.41, P=0.07), and 10-fold (R=0.46, P=0.05) and again using only negative tracts [leave-one-patient-out CV: R=0.35, P=0.12), 5-fold CV: (R=0.34, P=0.12), and 10-fold (R=0.42, P=0.07)). Repeating the leave-one-patient-out analysis 1,000 times after permuting motor progression values across subjects revealed the null-distribution for this experiment (FIG. 4E), which was centered around an R ˜0 (the R value of the unpermuted case, R=0.56 ranked significantly at p=0.039). Motor progression distributions for each randomization group from the ‘DBS in early-stage PD’ pilot trial are shown in FIG. 4D. Of note, motor progression scores for one-third of early DBS subjects were the same or better two years after baseline (5/14; FIG. 4D). A sensitivity analysis using ‘full UPDRS-III’ motor progression scores (blinded scores+unblinded rigidity score) produced a similar tractography profile and cross-validations as the primary analysis (leave-one-patient-out CV: R=0.49, P=0.04), 5-fold CV: (R=0.38, P=0.10), 10-fold CV (R=0.47, P=0.04)).

Relationship Between Motor Improvement and Stimulation Site. While the key focus of this study was to determine the relationship between stimulation site and slowing of motor progression (comparing 7-day OFF scores before and 2 years after surgery), it is important to compare these results with optimal stimulation sites associated with symptomatic motor improvements (comparing ON medication scores before surgery with scores while treated both with medication and DBS two years after surgery). The inventors felt it would be critical to determine whether the optimal sites and networks for these two distinct measures agree—or whether each would require stimulating a distinct spot/network.

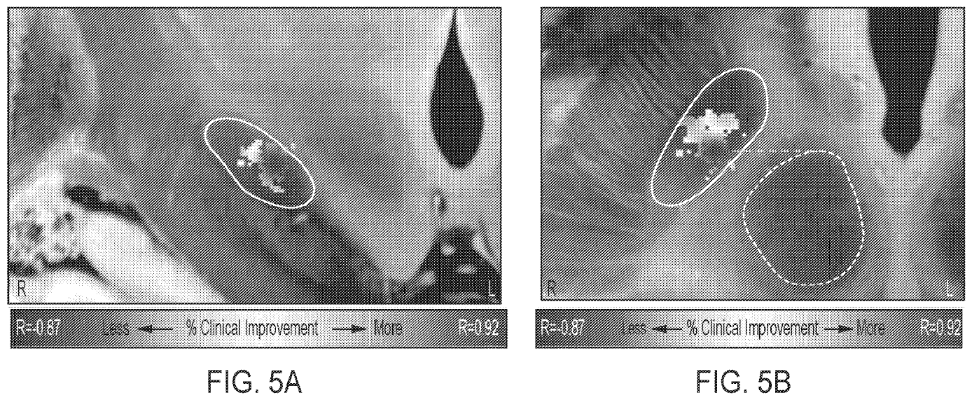

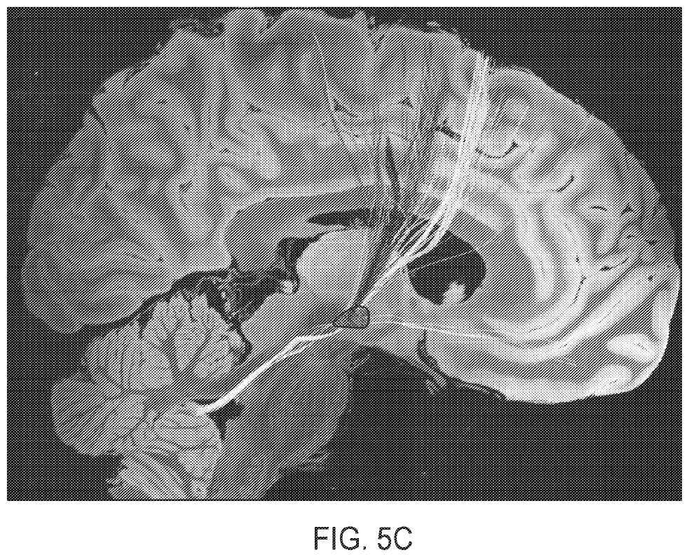

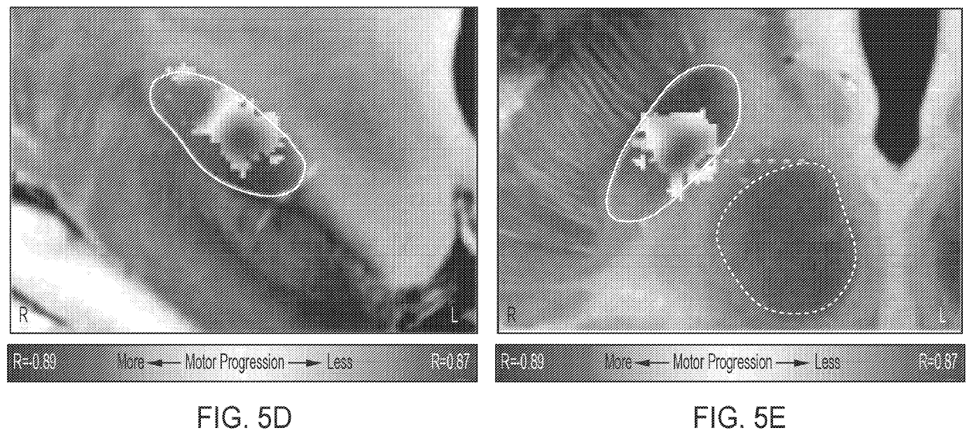

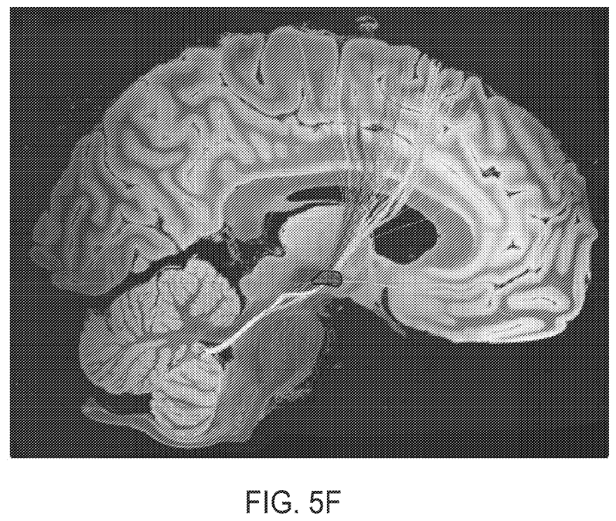

Therefore, the sweet spot analysis was repeated using motor improvement scores (UPDRS-III ON percent change from baseline to 24 months; FIGS. 5A-B) instead of motor progression scores (UPDRS-III 7-day OFF change from baseline to 24 months; FIGS. 3A-F, FIGS. 4A-E). Overall, the location associated with motor improvement and slowed motor progression distinctly overlapped at the subthalamic level (Euclidean distance: 0.12 mm). Indeed, the motor improvement sweet spot map peaked at +11.2, −13.7, −7.4 mm (MNI coordinates; with a peak Spearman's rank correlation coefficient of R=0.92), in close proximity to the motor progression sweet spot (FIGS. 3A-F). Expressed in functional (AC/PC) coordinates (Horn, Kuhn, et al., 2017), this would map to 11.08±0.82 mm lateral to, 1.93±0.60 mm posterior to, and 3.48±0.38 mm inferior to the midcommissural point. The fiber filtering analysis was also repeated using the motor improvement outcome. Results converged on a very similar network to the motor progression outcome results (FIG. 5C) and again showed strong and significant correlations (leave-one-patient-out CV: R=0.68, P<0.01), 5-fold CV: (R=0.62, P=0.01), and 10-fold (R=0.69, P=0.04)). Independent validation of positive and negative tracts revealed significant correlations when using positive tracts (leave-one-patient-out CV: R=0.70, P<0.01), 5-fold CV: (R=0.67, P<0.01), and 10-fold (R=0.72, P<0.01)) but not negative tracts (leave-one-patient-out CV: R=0.02, P=0.468, 5-fold CV: R=0.11, P=0.36, and 10-fold CV: R=0.10, P=0.36). To further explore (in)dependence of motor progression scores and symptomatic improvement outcomes (R=0.46, P=0.03) in relationship to our main results, we repeated the sweet spot and fiber filtering analyses on motor progression scores that were cleaned from motor improvement outcome scores (FIGS. 5D-F). This showed highly similar results, suggesting that the two scores did share additional, not the same, variance on group level.

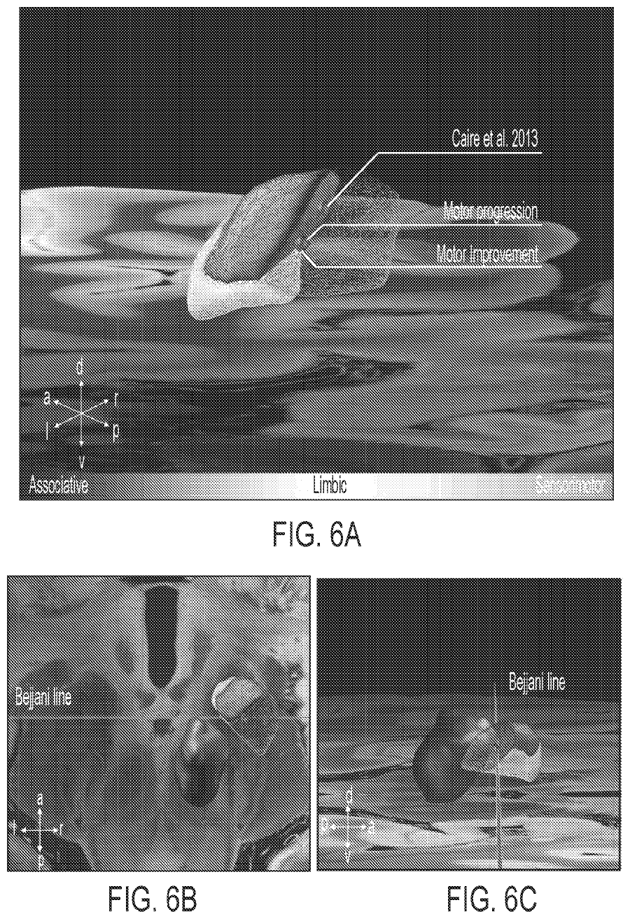

Comparison to Other Sweet Spots. To further characterize the anatomical relationship of the inventors' identified early PD sweet spots with respect to previously established anatomical boundaries and targets, the inventors searched the literature for established landmarks in DBS targeting. The search yielded two established landmarks, namely the Bejjani line (Bejjani et al., 2000) (identified on axial slices and constitutes a line connecting the anterior aspects of the red nucleus at its maximal diameter at the level of STN, which is commonly used as an anatomical reference in STN-DBS planning) and the metanalytical target by Caire et al. (Caire et al., 2013) (derived from 171 patients and has been associated with optimal outcome (Horn, Kuhn, et al., 2017)). The spatial relationship between previously published PD sweet spots in the literature and the motor progression and motor improvement sweet spots identified in this early-stage cohort is featured in FIGS. 6A-C. Comparison of target locations revealed intersection of both these targets and the target identified by Caire et al. (Caire et al., 2013) with the Bejjani line (Bejjani et al., 2000). The sweet spots associated with motor improvement and slowed motor progression in the present study, however, revealed variability in the mediolateral and ventrodorsal planes indicating a more ventral and lateral position of these targets with respect to the metanalystical target of advanced-stage PD by Caire et al. (Bejjani et al., 2000) (mean Euclidean Distance: 2.2±0.01 mm).

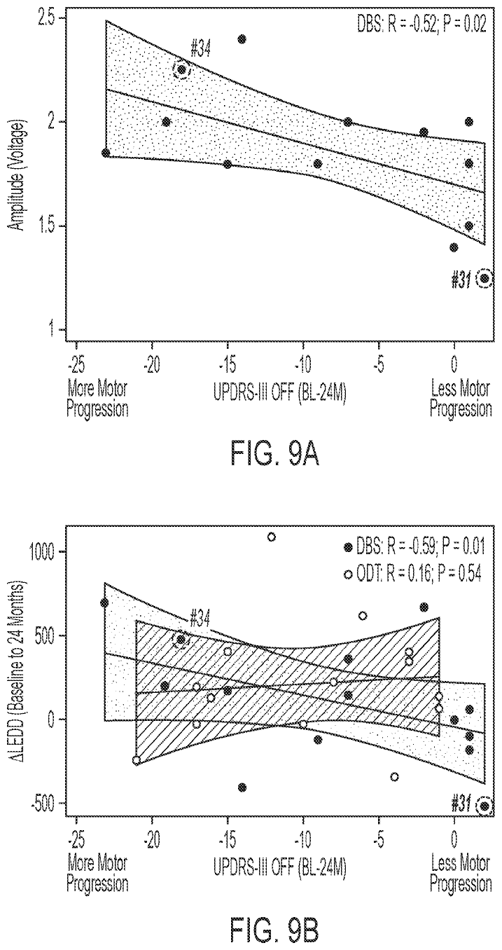

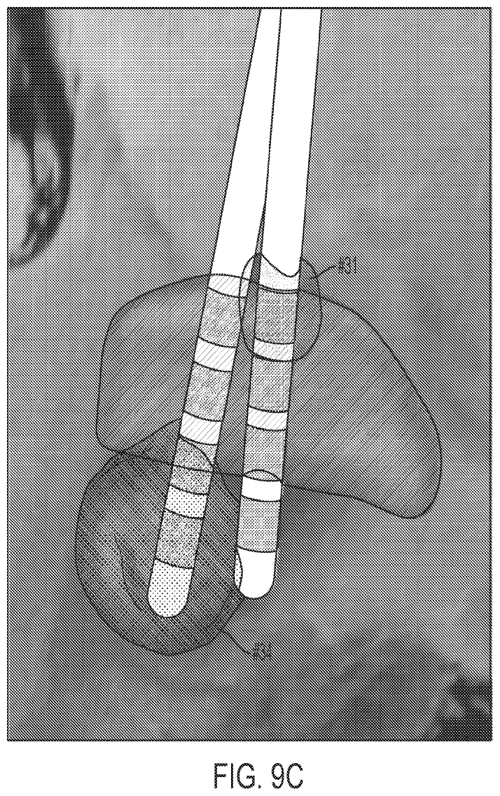

Relationships Between Motor Progression and PD Therapies. To explore whether slower motor progression across subjects who received STN-DBS could be explained by receiving more stimulation voltage and/or more PD medications, we assessed correlations between motor progression and the symptomatic therapies that were withdrawn during the seven-day washouts. Critically, lower stimulation amplitude at 24 months correlated with slower motor progression (R=−0.52, P=0.02; FIG. 9A), and there was also a significant correlation between greater reductions in LEDD (change from baseline to 24 months) and slower motor progression for DBS subjects (R=−0.59, P=0.01; FIG. 9B). There was no relationship between change in LEDD and motor progression for subjects randomized to ODT (R=0.16, P=0.54; FIG. 9B). These results, in combination with our electrode localization analyses, suggest that stimulation location, not the amount of PD therapy given, drives motor benefit.

### Example 3—Discussion

Results from the DBS in early PD pilot clinical trial provided class II evidence that early DBS slows rest tremor progression and decreases the risk of disease progression and polypharmacy (M. Hacker et al., 2018; M. L. Hacker et al., 2020). To better understand these findings, this study evaluated individual subject motor progression and explored the relationship between stimulation regions and motor progression.

There are four main conclusions that can be drawn from this study. First, untreated motor symptoms did not progress over 2 years for one-third of subjects randomized to DBS+ODT, compared to progressing for all subjects randomized to ODT. Second, slowed motor progression significantly correlated with stimulating cortical input fibers from M1 and supplementary motor area (but not the pre-SMA). Third, the optimal location and tracts for slowing motor progression in early PD were very similar to those associated with early PD symptomatic motor improvement. In other words, the network associated with optimal clinical response was also associated with slowing of motor progression. Finally, the location and tracts identified for optimal benefit in early-stage PD were similar to previously-reported efficacious locations from studies of patients who received DBS in more advanced stages of PD (Akram et al., 2017; Horn, Li, et al., 2019; Horn, Reich, et al., 2017).

From baseline to 2 years, untreated motor symptoms for one-third of subjects randomized to receive early DBS did not progress; in fact, scores were improved from baseline for 4 out of 5 top responders. By contrast, motor symptoms worsened for every subject randomized to ODT. This striking finding raises important new questions: Why did these early DBS subjects have such robust slowing of motor progression? And, importantly, how could future early DBS trials increase the number of participants that experience slowed motor progression?

The motor progression outcome was used post-hoc to generate the DBS sub-groups, and it is, therefore, not surprising to see such a large difference between the top responders and the typical responders in FIG. 1A. However, this sub-grouping did not influence the statistics of subsequent imaging analyses, and separating the DBS+ODT group based on this responder analysis revealed independent key differences between the groups: top responders required significantly lower levels of both medications (FIG. 1B) and stimluation (FIG. 1C) than typical responders, while still achieving slower motor progression. Since optimal electrode placement is not only associated with reduced required therapy (medications and stimulation) but is also a key source of variance among DBS patients (Caire et al., 2013; Frizon et al., 2018; Horn, Li, et al., 2019), this finding motivated additional exploration into electrode placement of this unique cohort.

The optimal stimulation sites identified here map closely to previously-published sweet spots associated with symptom improvement in advanced-stage PD (Akram et al., 2017; Bot et al., 2018; Horn, Li, et al., 2019) (for a review see (Horn, 2019)). This posteriorlateral STN location aligns anteriorly/posteriorly with the anterior border of the red nucleus, known as the Bejjani line, which is commonly used for surgical targeting of STN-DBS for PD (Bejjani et al., 2000). Since DBS is intendend to be used throughout PD progression, it is a key finding that optimal targets for PD symptom improvements align with those for motor progression and that these targets also align between early and more advanced stages of PD. This suggests that precise surgical targeting to this established location for advanced PD is expected to not only also provide symptomatic benefit in early-stage PD but also potentially slow motor symptom progression. Importantly, this slowed motor progression does not require stimulating a distinct network or site.

While targeting focal locations of the STN (defined by local relationships to landmarks) is commonly used for surgical planning (Bejjani et al., 2000), there is an increasing paradigm shift in conceptualizing the DBS benefit in relationship to the global networks modulated by DBS (Horn, Reich, et al., 2017; Lozano & Lipsman, 2013b). Motor benefit in early-stage PD strongly correlated with fiber tracts from the hyperdirect pathway, specifically those connecting from M1 and SMA, but not pre-SMA, to the STN. Given prior associations of the hyperdirect pathway with symptomatic motor improvement for STN-DBS patients with advanced-stage disease (Akram et al., 2017; Avecillas-Chasin & Honey, 2020; Horn, Reich, et al., 2017), it is not surprising that these fiber tracts would also impart motor benefit for early-stage PD patients. Evidence continues to accumulate that connections from M1 improve tremor while those from SMA improve hypokinetic symptoms (Akram et al., 2017; Sobesky et al., 2022) which confirms knowledge established by early lesional work (Hassler et al., 1960). There is less clarity on the exact division between hyperdirect input from SMA vs pre-SMA in DBS for PD. These neighboring cortical regions are not directly connected to each other (Akkal et al., 2007), and they also show notable differences in their functional roles and connectivity profiles (Kim et al., 2010). Interestingly, here, modulating hyperdirect tracts originating from pre-SMA were negatively associated with both slowing motor progression and clinical improvements. The same negative association applied to fibers of passage corresponding to the non-decussating dentatothalamic tract. It is uncertain whether these negative tracts play a causal role or result from spurious correlations (i.e., patients with poorer outcomes happened to modulate these connections by chance) but the causal ingredient was that they did not modulate the beneficial connections. Notably, leaving out negative tracts or positive tracts (and repeating the analysis) led to inferior results for slowing motor progression, suggesting that both modulating positive tracts and not modulating negative tracts could play a role in mediating effects. Critically, this was not the case for tracts associated with symptom improvements, where only stimulation overlaps with positive tracts showed significant predictive value (while reanalyzing the data using only the negative tracts did not yield significant results).

Preclinical studies demonstrate the importance of early DBS intervention (Maesawa et al., 2004; Musacchio et al., 2017; A. L. Spieles-Engemann et al., 2010; Temel et al., 2006), and the rationale to intervene early is further underscored by post-mortem data showing that 50% of putaminal denervation occurs by the time PD is diagnosed, rising sharply to 90% by 4 years disease duration (Kordower et al., 2013). Therefore, while numerous studies have evaluated the relationship between electrode placement and motor improvement in PD (Akram et al., 2017; Blomstedt et al., 2012; Bot et al., 2018; Butson et al., 2006; Caire et al., 2013; Horn, Li, et al., 2019; Maks et al., 2008; Plaha et al., 2006), this is the first study evaluating how stimulation location could affect disease progression.

The inventors' results suggest that targeting M1 and SMA hyperdirect tracts to the STN in early-stage PD is associated with slowing of motor progression. It is important to clarify that these results are based on a post-hoc analysis and small sample and that results do not provide evidence of neuroprotection, which cannot be shown without a validated biomarker. Modifying the course of PD by slowing motor progression, however, does represent disease modification (Vijiaratnam et al., 2021). It is currently unclear how such a disease-modifying effect might occur, but work from others may shed light on potential mechanisms. Long-term plasticity with DBS in the sensorimotor network is suggested by prolonged beta band attenuation after DBS withdrawal from two longitudinal studies (Chen et al., 2020; Trager et al., 2016). Since the hyperdirect pathway may be the prominent source of high beta activity in STN (Oswal et al., 2021), the association of this pathway with slowing early PD motor progression is intriguing. However, it is likely that indirect projections pointing to the same subthalamic loop would play a similar role but went undetected by the inventors' analysis. Anatomically, these correspond to comb fibers that are not visible on dMRI-based tractography datasets (Horn, Ewert, et al., 2019; Noecker et al., 2021). Chronic sensing-enabled DBS systems now permit longitudinal electrophysiological recordings, and future studies exploring how electrode location affects beta band activity as well as motor progression could help elucidate potential mechanisms. Additionally, preclinical studies point to brain-derived neurotrophic factor (BDNF) signaling as a potential mediator of these effects: STN stimulation increases BDNF in the striatum, substantia nigra, and M1 cortex (AL L Spieles-Engemann et al., 2011) and BDNF signaling via its high-affinity receptor tropomyosin-related kinase type B (trkB) is associated with the neuroprotective and symptomatic efficacy of STN-DBS (D. Fischer et al., 2017). Increases of this prominent neurotrophic growth factor could promote neuron survival, maintenance of the cortical-basal ganglia circuitry or even decrease alpha-synuclein (D. L. Fischer & Sortwell, 2019).

Limitations of this retrospective analysis should also be discussed. Patient-specific tractography data were not collected in the pilot trial, and instead, normative connectome data were used. Although this approach lacks patient-specific anatomical features, test-retest studies show that larger fractions of differences observed in individualized tractography data are due to noise (and not true anatomical variations across patients) (Petersen et al., 2017) and that, for instance, the effect of the MRI scanner can be larger than the one of the patient, in DBS (Jakab et al., 2016). Furthermore, normative connectome data has been shown to yield similar results to patient-specific data in PD (Wang et al., 2021). Future studies collecting patient-specific connectivity data may be able to explain more variance. Similarly, reconstruction of electrode placements and aggregating them to a common space that allows comparisons leads to bias due to imaging resolution, so reconstructed electrodes do not exactly match reality. To this end, a modern pipeline was used that was specifically created for this task, using concepts such as brain shift correction, multispectral normalizations (Horn, Li, et al., 2019), phantom-confirmed electrode localizations (Husch et al., 2018), and a validated segmentation framework (Ewert et al., 2019). Moreover, recently, bias introduced by the user localizing Lead-DBS, as well as postoperative imaging modality was quantified and remained below the magnitude of imaging resolution (Lofredi et al., 2022). Since this is the only cohort implanted with DBS in early-stage PD and also the only study evaluating a motor progression outcome, the inventors were unable to independently validate findings in a separate cohort. These results are therefore hypothesis-generating and should be prospectively tested in future studies. One key strength of this study is the meticulously collected longitudinal blinded clinical ratings of the UPDRS-11T motor examination (ON therapy and 7 days OFF therapy); however, blinded ratings inherently rely on videotaped recordings of the exam for scoring, which precludes evaluation of rigidity. While symptomatic effects of DBS wash out within hours (Temperli et al., 2003), lingering symptomatic effects from PD medications can persist longer than 7 days. Symptomatic effects of levodopa, for example, can last weeks or even months (Hauser & Holford, 2002; Nutt et al., 1997; Olanow et al., 1995). However, for a clinical trial evaluating DBS in early-stage PD, a 7-day therapeutic washout strikes an appropriate balance between scientific rigor (i.e., how long is needed to wash out symptomatic effects of the intervention being tested?) and reasonable burden to study participants (i.e., what is practically and ethically feasible to ask early-stage PD patients to endure?). Importantly, participants randomized to ODT received more medications throughout the trial (David Charles et al., 2014) and are therefore expected to have more lingering symptomatic effects compared to DBS+ODT subjects. Of relevance in this study, the 5 top DBS+ODT responders whose motor symptoms did not progress over 2 years were taking less medication than both the typical DBS+ODT responders and the ODT control group.

This study analyzed the relationship between electrode location and motor outcomes from the DBS in early-stage PD pilot clinical trial. These results suggest that DBS electrodes stimulating the posteriorlateral STN, specifically the zones of the nucleus receiving input from M1 and SMA, can slow motor progression in early-stage PD. This finding must be prospectively confirmed in larger study, and the FDA has approved a planned multicenter, phase 3 clinical trial evaluating DBS in early-stage PD.

TABLE 1
Demographics and Baseline Characteristics

| | | DBS + ODT | | |
|---|---|---|---|---|
| | | Full | Typical | Top |
| | ODT | Cohort | Responders | Responders |
| | 14 | 14 | 9 | 5 |
| Sex, Male | 12 (86%) | 13 (93%) | 8 (89%) | 5 (100%) |
| Age | | | | |
| Years | 60.7 ± 6.6 | 60.9 ± 6.9 | 62.8 ± 7.5 | 57.4 ± 4.0 |
| Range | 50.2-69.8 | 52.2-74.5 | 54.0-74.4 | 52.3-62.5 |
| Medication Use | | | | |
| Mean Duration | 2.0 ± 1.0 | 2.6 ± 1.9 | 2.1 ± 1.4 | 3.6 ± 2.5 |
| (Years) | | | | |
| Mean LEDD (mg) | 490.7 ± 216.2 | 429.9 ± 314.1 | 358.6 ± 350.1 | 558.2 ± 208.5 |
| Handedness, Right | 12 (86%) | 14 (100%) | 9 (100%) | 5 (100%) |
| Side of Onset, Right | 7 (50%) | 10 (71%) | 6 (67%) | 4 (80%) |
| UPDRS-III^{~} | | | | |
| ON | 21.3 ± 9.2 | 23.1 ± 12.6 | 27.1 ± 11.4 | 16.0 ± 12.6 |
| OFF (7 Days) | 29.5 ± 8.7 | 28.0 ± 10.2 | 29.9 ± 9.0 | 24.6 ± 12.4 |
| Total UPDRS ON | 33.4 ± 13.3 | 35.0 ± 16.3 | 40.8 ± 14.8 | 24.6 ± 14.6 |
| PD Subtype^{#} | | | | |
| Tremor-Dominant | 9 | 8 | 5 | 3 |
| PIGD-Dominant | 2 | 3 | 2 | 1 |
| Mixed | 3 | 3 | 2 | 1 |

LEDD, levodopa equivalent daily dose
ODT, optimal drug therapy
PIGD, Postural Instability and Gait Disorder
UPDRS, Unified Parkinson's Disease Rating Scale
^{~}Excludes rigidity
^{#}OFF scores were used to calculate PD phenotype using method described by Stebbins et al. and previously used in Hacker et al.

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Starr, P. A., Stereotact. Funct. Neurosurg. 2002, 79: 118-145.
Hutchinson et al., Ann. Neurol.1998 44(4): 622-8.
Gross et al., Mov. Disord. 2006, 21(14): S259-S283.
Charles et al., Parkinsonism & Related Disorders. 2014; 20(7):731.
Spieles-Engemann et al., Neurobiology of Disease. 2010; 39(1):105.
Kahn et al., Journal of Neurology, Neurosurgery, and Psychiatry 2012; 83(2):164-170.
Plaha et al., Brain 2006; 129(7):1732-1747.
Caire et al., Stereotact Funct Neurosurg. 2009; 87: 337-347.
Akkal et al., (2007). Journal of Neuroscience, 27(40), 10659-10673.
Akram et al., (2017). NeuroImage, 158(January), 332-345.
∪ström, M., Diczfalusy, E., Martens, H., & Wirdell, K. (2015). IEEE Trans Biomed Eng, 62(2), 664-672.
Avants, B. B., Epstein, C. L., Grossman, M., & Gee, J. C. (2008). Medical Image Analysis, 12(1), 26-41.
Avants, Brian B., Tustison, N. J., Song, G., Cook, P. A., Klein, A., & Gee, J. C. (2011). NeuroImage, 54(3), 2033-2044.
Avecillas-Chasin, J. M., & Honey, C. R. (2020). Neurosurgery, 86 (4), E387-E397.
Bejjani, B. P., Dormont, D., Pidoux, B., Yelnik, J., Damier, P., Arnulf, I., Bonnet, A. M., Marsault, C., Agid, Y., Philippon, J., & Cornu, P. (2000). Journal of Neurosurgery, 92(4), 615-625.
Blomstedt et al., (2012). Parkinsonism and Related Disorders, 18(10), 1062-1066.
Blomstedt et al., (2018). Journal of Neurology, Neurosurgery & Psychiatry, 89 (7), jnnp-2017-317219.
Bot et al., (2018). Journal of Neurology, Neurosurgery & Psychiatry, 2018; 89: 493-8.
Butson, C. R., Maks, C. B., & McIntyre, C. C. (2006). Sources Clinical Neurophysiology, 117(2), 447-454.
Caire, F., Ranoux, D., Guehl, D., Burbaud, P., & Cuny, E. (2013). Acta Neurochirurgica, 155(9), 1647-1654.
Camalier et al., (2014). Frontiers in Neurology, 5.
Charles et al., (2012). Journal of Parkinson's Disease, 2, 215-223.
Charles et al., (2014). Parkinsonism and Related Disorders, 20(7), 731-737.
Charles et al., (2002). Neurology, 59(6), 932-934.
Chen et al., (2020). Brain Stimulation, 13(6), 1784-1792.
Deuschl et al., (2006). New England Journal of Medicine, 355(9), 896-908.
Ewert et al., (2019). NeuroImage, 184(April 2018), 586-598.
Ewert et al., (2018). NeuroImage, 170 (May 2017), 271-282.
Fischer et al., (2017). The Journal of Neuroscience, 37(28), 6786-6796.
Fischer, D. L., & Sortwell, C. E. (2019). Movement Disorders, 34(1), 22-34.
Fonov et al., (2011). NeuroImage, 54(1), 313-327.
Friston et al., (2004). Hum. Brain Mapp. 2, 189-210.
Frizon et al., (2018). Journal of Neurosurgery, 130(6), 1841-1846.
Hacker et al., (2018). Neurology, 91(5), e463-e471.
Hacker et al., (2015). Parkinsonism and Related Disorders, 21(10), 1177-1183.
Hacker et al., (2020). Neurology, 95(4), E393-E401.
Hariz, M. I., Blomstedt, P., & Zrinzo, L. (2010). Deep brain stimulation between 1947 and 1987: the untold story. Neurosurgical Focus, 29(2), E1.
Hassler, R., Riechert, T., Mundinger, F., Umbach, W., & Ganglberger, J. A. (1960). Brain, 83(2), 337-350.
Hauser, R. A., & Holford, N. H. G. G. (2002). Movement Disorders, 17(5), 961-968.
Horn, A. (2019). Current Opinion in Neurology, 32(4), 511-520.
Horn et al., (2019). Neurology, 92(14), e1663-e1664.
Horn, A., & Kuhn, A. A. (2015). NeuroImage, 107, 127-135.
Horn, A., Kühn, A. A., Merkl, A., Shih, L., Alterman, R., & Fox, M. (2017). NeuroImage, 150 (February), 395-404.
Horn et al., (2019). NeuroImage, 184 (August 2018), 293-316.
Horn et al., (2022). Proc Nail Acad Sci USA 2022; 119: 1-11.
Horn et al., (2017). Annals of Neurology, 82(1), 67-78.
Husch, A., V. Petersen, M., Gemmar, P., Goncalves, J., & Hertel, F. (2018). NeuroImage: Clinical, 17(May 2017), 80-89.
Jakab et al., (2016). Frontiers in Neuroanatomy, 10(July), 1-15.
Jakobs, M., Fomenko, A., Lozano, A. M., & Kiening, K. L. (2019). EMBO Molecular Medicine, 11(4), 1-18.
Johnson, H., Harris, G., Williams, K. (2007). BRAINSFit: Mutual Information Registrations of Whole-Brain 3D Images, Using the Insight Toolkit. The Insight Journal, Vol. 180-2007 July-December https://doi.org/10.54294/hmb052.
Kahn et al., (2011). Deep brain stimulation in early stage Parkinson's disease: operative experience from a prospective randomised clinical trial. Journal of Neurology, Neurosurgery & Psychiatry, 83(2), 164.
Kahn et al., (2012). Journal of Neurology, Neurosurgery, and Psychiatry, 83(2), 164-170.
Kim, et al., (2010). NeuroImage, 49(3), 2375-2386.
Kordower et al., (2013). Brain, 136(8), 2419-2431.
Krauss et al., (2020). Nature Reviews Neurology.
Li et al., (2020). Biological Psychiatry, 11(10), 701-713.
Lofredi et al., (2022). NeuroImage, 262 (February), 119552.
Lozano, A. M., & Lipsman, N. (2013a). Neuron, 77(3), 406-424.
Lozano, A. M., & Lipsman, N. (2013b). Neuron, 77(3), 406-424.
Maesawa et al., (2004). Journal of Neurosurgery, 100(4), 679.
Maks et al., (2008). Journal of Neurology, Neurosurgery & Psychiatry, 80(6), 659.
Middlebrooks et al., (2020). American Journal of Neuroradiology, 41(9), 1558-1568.
Musacchio et al., (2017). Annals of Neurology.
Neudorfer et al., (2022). Annals of Neurology, 91(5), 613-628.
Noecker et al., (2021). StimVision v2: Neuromodulation, 24(2), 248-258.
Nutt, J. G., Carter, J. H., Van Houten, L., & Woodward, W. R. (1997). Annals of Neurology, 42(3), 349-355.
Olanow et al., (1995). Annals of Neurology, 38(5), 771-777.
Oswal et al., (2021). Nature Communications, 12(1), 1-14.
Petersen et al., (2017). Journal of Neurosurgery, 126(5), 1657-1668.
Plaha, P., Ben-Shlomo, Y., Patel, N. K., & Gill, S. S. (2006). Brain, 129(7), 1732-1747.
Schuepbach et al., (2013). New England Journal of Medicine, 368(7), 610.
Sobesky et al., (2022). Brain, 145(1), 251-262.
Spieles-Engemann, et al. (2010). Neurobiology of Disease, 39(1), 105-115.
Spieles-Engemann et al., (2011). Journal of Parkinson's Disease, 1(1), 123-136.
Stebbins et al., (2013). Movement Disorders, 28(5), 2012-2014.
Temel et al., (2006). Brain Research, 1120(1), 100-105.
Temperli et al., (2003). Neurology, 60(1), 78-81.
Tomlinson, C. L., Stowe, R., Patel, S., Rick, C., Gray, R., & Clarke, C. E. (2010). Movement Disorders, 25(15), 2649-2685.
Trager et al., (2016). Neurobiology of Disease, 96, 22.
Treu et al., (2020). NeuroImage, 219(January), 117018.
Vijiaratnam, N., Simuni, T., Bandmann, O., Morris, H. R., & Foltynie, T. (2021). The Lancet Neurology, 20(7), 559-572.
Vorwerk, J., Magyari, L., Ludewig, J., Oostenveld, R., & Wolters, C. H. (2013). First International Conference on Basic and Clinical Multimodal Imaging BaCI, Geneva, Sw.
Wang et al., (2021). NeuroImage, 224(August 2020), 117307.
Weaver et al., CSP 468 Study Group. (2012). Neurology, 79(1), 55-65.
Welter et al., (2002). Brain, 125(3), 575-583.

1. A method of placing a deep brain stimulation (DBS) electrode into a patient having early-stage Parkinson's Disease (PD) comprising:
(a) mapping the patient's brain to determine a location for DBS electrode placement by identifying (i) fiber tracts from the supplementary motor area and/or primary motor area to the subthalamic nucleus (STN) of the patient; and (ii) fiber tracts from the pre-supplementary motor area to the STN of the patient; and
(b) implanting a DBS electode to target (a)(i) and avoid targeting (a)(ii).

2. A method of placing and programming a deep brain stimulation (DBS) electrode into a patient having early-stage Parkinson's Disease (PD) comprising:
(a) implanting a DBS electode target the subthalamic nucleus (STN) of the patient;
(b) mapping the patient's brain to determine programming of said DBS electrode by identifying (i) fiber tracts from the supplementary motor area and/or primary motor area to the subthalamic nucleus (STN) of the patient; and (ii) fiber tracts from the pre-supplementary motor area to the STN of the patient, wherein the DBS electrode is programmed to stimulate (i) and avoid stimulating (ii).

3. The method of claim 1, further comprising treating said patient by delivering an electrical current through said DBS electrode, such as by continous delivery, patient modulated delivery, or by adaptive delivery based on patient parameters.

4. The method of claim 1, wherein said patient is a male human patient.

5. The method of claim 1, wherein said patient is a female human patient.

6. The method of claim 1, wherein said patient is a non-human mammalian subject.

7. The method of claim 1, wherein DBS is performed more than once, such as on a chronic basis.

8. The method of claim 1, further comprising treating said patient with a second PD therapy.

9. The method of claim 8, wherein said second PD therapy is administered prior to STN-DBS.

10. The method of claim 8, wherein said second PD therapy is administered at the same time as STN-DBS.

11. The method of claim 8, wherein said second PD therapy is administered after STN-DBS.

12. The method of claim 8, wherein said second PD therapy is selected from levodopa, optionally in combination with a DOPA decarboxylase inhibitor (carbidopa, benserazide) or a COMT inhibitor (tolcapone, entacapone), a dopamine agonist (e.g., apomorphine, bromocriptine, pergolide, pramipexole, ropinirole, piribedil, cabergoline, apomorphine, lisuride), an MAO-B inhibitor (e.g., safinamide, selegiline, rasagiline), amantadine, an anticholinergics cholinesterase inhibitor, and lesional surgery, or combinations thereof.

13. The method of claim 1, wherein STN-DBS results in one or more of slowing of motor symptom progression, stopping motor symptom progression, and/or reversing motor symptom progression.

14. The method of claim 1, wherein STN-DBS results in one or more of lower stimulation parameters, less need for post-operative dopaminergic medication, and/or less development of levodopa associated dyskinesia or other motor fluctuations.

15. The method of claim 1, wherein step (a) comprises:
identifying the patient-specific location of the tracts defined in (a)(i) and (a)(ii) from a normative connectome by using inverse normalization to warp the tracts from the template space into the patient's brain space; or
wherein step (a) comprises identifying the patient-specific location of the tracts defined in (a)(i) and (a)(ii) from a normative connectome by normalizing the patient's brain to the template space which includes the tracts.

16. The method of claim 1, wherein step (a) comprises utilizing patient-specific tractography data collected from diffusion-weighted brain imaging of the patient's brain to identify (a)(i) and (a)(ii) using the following regions of interest (ROI):
1) from the supplementary motor area projecting to the STN;
2) from the primary motor area projecting to the STN; and
3) from the pre-SMA projecting to the STN.

17. The method of claim 1, further comprising performing a post-operative scan of the patient's brain.

18. The method of claim 2, wherein step (b) comprises:
identifying the patient-specific location of the tracts defined in (b)(i) and (b)(ii) from a normative connectome by using inverse normalization to warp the tracts from the template space into the patient's brain space; or
wherein step (b) comprises identifying the patient-specific location of the tracts defined in (b)(i) and (b)(ii) from a normative connectome by normalizing the patient's brain to the template space which includes the tracts.

19. The method of claim 2, wherein step (b) comprises utilizing patient-specific tractography data collected from diffusion-weighted brain imaging of the patient's brain to identify (b)(i) and (b)(ii) using the following regions of interest (ROI):
1) from the supplementary motor area projecting to the STN;
2) from the primary motor area projecting to the STN; and
3) from the pre-SMA projecting to the STN.

20. The method of claim 18, determining whether said DBS electrode has achieved intended preoperative targeting of (b)(i) and non-targeting of (b)(ii).

21. The method of claim 18, wherein said DBS electrode comprises a plurality of contacts or segments and said method further comprises determining which contact(s) or segment(s) provide(s) the maximal stimulation of (b)(i) and avoids (b)(ii).

22. The method of claim 18, wherein said DBS electrode comprises a plurality of contacts or segments, and said method further comprises determining a field shape for said contact(s) or segment(s) that provide(s) maximal stimulation of (b)(i) and that avoids (b)(ii).

23. The method of claim 2, wherein said DBS electrode comprises a plurality of contacts or segments and said method further comprises determining which contact(s) or segment(s) provide(s) the maximal stimulation of (b)(i) and avoids (b)(ii).

24. The method of claim 2, wherein said DBS electrode comprises a plurality of contacts or segments, and said method further comprises determining a field shape for said contact(s) or segment(s) that provide(s) maximal stimulation of (b)(i) and that avoids (b)(ii).

25-29. (canceled)

30. A computer implemented method for identifying deep brain stimulation (DBS) electrode placement locations for DBS treatment of a patient having early-stage Parkinson's Disease (PD) comprising the steps of:
(a) receiving brain image data for the patient;
(b) processing the brain image data to identify fiber tracts (i) from the supplementary motor area and/or primary motor area to the subthalamic nucleus (STN) of the patient, and (ii) from the pre-supplementary motor area to the STN of the patient; and
(c) generating an electrode placement map for the treatment of the patient using DBS, such that the implanted electrodes will target (a)(i) and avoid targeting (a)(ii).

31. A computer implemented method for identifying deep brain stimulation (DBS) electrode placement programming for DBS treatment of a patient having early-stage Parkinson's Disease (PD) comprising the steps of:
(a) receiving brain image data for the patient;
(b) processing the brain image data to identify fiber tracts (i) from the supplementary motor area and/or primary motor area to the subthalamic nucleus (STN) of the patient, and (ii) from the pre-supplementary motor area to the STN of the patient; and
(c) generating an electrode programming map for the treatment of the patient using DBS, such that the implanted electrodes will target (a)(i) and avoid targeting (a)(ii).

32-34. (canceled)
